(19) United States
(12) Patent Application Publication
(10) Pub. No.: US 2026/0273076 A1
(43) Pub. Date: Sep. 17, 2026

(54) PHARMACEUTICAL COMPOSITIONS AND METHOD OF TREATMENT OF METASTATIC AND OTHER CANCERS

(71) Applicant: Favorite Pharmaceuticals, Edison, NJ (US)

(72) Inventors: Robert G. L. SHORR, Edison, NJ (US); Jeffrey Schwartz, Edison, NJ (US); Jacques Y. ROBERGE, Princeton, NJ (US)

(73) Assignee: Favorite Pharmaceuticals, Edison, NJ (US)

(21) Appl. No.: 19/470,369

(22) PCT Filed: Apr. 2, 2024
(86) PCT No.: PCT/US2024/022660

§ 371 Date: Sep. 29, 2025

Related U.S. Application Data
(60) Provisional application No. 63/493,826, filed on Apr. 3, 2023.

(51) Int. Cl. A61K 47/60; A61K 47/54; A61P 35/00

(52) U.S. Cl. CPC A61K 47/60; A61K 47/542; A61P 35/00

(57) ABSTRACT

Methods of causing mammalian cell death useful in the treatment of cancer are disclosed. The methods include contacting a mammalian cell with a polymer conjugate containing a cellular uptake promoter, such as a fatty acid or nutrient or metabolite which can be taken up into a cancer cell from exogenous sources, covalently linked to a colloid polymer, such as polyethylene glycol (PEG) under conditions sufficient to allow the polymer conjugate to be taken up by the cell. Once cellular uptake has been achieved, the colloid polymer portions of the conjugates increase the intracellular oncotic or osmotic pressure of the cell, causing cell membrane disruption and death of the mammalian cell. The conjugate remains substantially intact after cellular uptake as a result of the linkage being substantially resistant to intracellular cleavage.

### FIELD OF THE INVENTION

The present invention is directed to anti-cancer polymer and colloid conjugates useful for selective disruption of cell and organelle phase-protein organization, membranes, and their integrity as well as metabolic events associated with diverse cancer cell types.

### BACKGROUND OF THE INVENTION

Heterogeneity of cancer cell types among disease indications, patients, and subpopulations, and even within the cancer burden of individual patients makes cancer an extremely difficult disease to manage. Novel chemical class agents with novel mechanisms of action for the killing of the most difficult to treat, heterogeneous range of cancer cell types is considered an urgent need. For example, without limitation, those cells in the circulation (cancer causing stem cells and facilitating immune cell populations), and elsewhere, leading to metastatic disease. Such cell types are typically unable to be effectively treated by current methods. Further, tumors considered inoperable or so poorly vascularized so as to minimize drug exposure. All leaving patients with the poorest projected survival outcome. Despite being treated with multiple therapeutic modalities and chemotherapeutic and other cancer targeted therapies and drugs. Including, the most recent immune oncology approaches and drugs.

The most desirable novel agents must be sufficiently broadly selective for the diverse cancer cell types without harming healthy cells, yet not so specific as to be ineffective against subpopulations among heterogeneous cell groups such that refractory resistant disease and metastasis emerge by natural selection and changes due to environmental factors within a tumor mass. In the most preferred embodiments of the invention, therapeutic agents are effective against the majority if not all, cancer- and cancer-causing cell types within a patient, even during disease progression, and sufficiently safe for longer-term, even chronic, use. The intention is to maximally increase overall survival by preventing recurrent or refractory disease emergence.

All cancer indications have specific metabolic processes requiring unique nutritional needs. Some aspects of cancer's unique metabolic phenotypes are more frequently observed among diverse cancer cell types than others. The requirement for particular more preferred essential nutrients can be characteristic of disease indications, macro and microenvironment and a reflection of the heterogeneity of cells being studied ex vivo or cancer cells in patients with increasing diversity occurring during disease progression (Weber and Winkle Review Frontiers in Oncology December 2016). Suggesting that tracking of patient derived cells for sensitivity to therapies given at appropriate intervals and testing for “most likely to be effective” drugs or combinations of drugs and treatment modalities is more likely to prolong survival and quality of life than “shots in the dark” protocol changes. This is especially the case for solid tumors where infiltrating circulating cells evolve and participate in metastasis formation (Kalikaki A, Politaki H, Souglakos J, Apostolaki S, Papadirnitraki E, Georgoulia N, et al. (2014) KRAS Genotypic Changes of Circulating Tumor Cells during Treatment of Patients with Metastatic Colorectal Cancer. PLoS ONE 9(8) and Marx V. Tracking metastasis and tricking cancer. Nature. 2013 Feb. 7:494(7435):133-6. In the treatment of solid cancers, elimination of invasive circulating tumor cells and clusters containing immune cells as well as disrupting other contributors to metastatic disease is of the highest priority. Currently, many invasive circulating tumor cells and other cells that contribute to formation of metastatic nodules are not effectively treated with current standards of care.

Many ex-vivo and even in-vitro models of disease are in-adequate in providing drug screening data sufficient to be patient predictive. Personalized medicine using patient derived cells and tissues are being studied for this purpose, and the identification of new drug targets and biomarkers. Substantial differences between solid tumors and cancers of the blood have been reported Rashkavan and Ferrando, Genes Dev. 2019 Nov. 1; 33(21-22): 1460-1474. Further emphasizing the need for the screening of potentially new therapeutic agents with novel mechanisms of action for initial treatment of patients and selection of therapeutics in the face of disease progression or relapse as cell populations change.

At the phenotypic level, some shared features of cancer metabolism may be observed among heterogeneous cells. The Warburg Effect is well known and associated with elevated needs for glucose, fatty acids, lipids, glutamine, and other amino acids. Additional changes more characteristic of specific cell types, even within a patient's own tumors and cancer cells circulating in the blood may be present and evolve with treatment and progression. Some cells are even capable of switching metabolic pathways according to levels of oxygen and nutrients available. The ability to use alternative fuel and nutrient sources and other metabolic pathways not only contributing to metastatic disease, but even may allow escape from metabolic disrupting and other drugs. The essential nature of a nutrient or cofactor suggests that the necessary receptors and transporters to cancer cells and organelles for required metabolism are present, may be overexpressed, or even unique to diseased cells. However, the receptors and transporters for essential nutrients may be saturable, located in the blood or associated with cancer cell membranes, placing restrictions on dosage forms and optimal dosing rates and protocols. This is especially true when drugs are used in combinations as competition between drugs or even excipients used in formulation may affect overall biodistribution and pharmacokinetics.

Cells that can be killed by a particular treatment and eradicated from a patient may be replaced by an alternative cell type, not responsive to the initial treatment, and becoming the dominant cell type by natural selection. For drugs intended for metabolic disruption, whether as a single agent or in combination with other drugs a further complication may be asynchronous rates of cell division and any cell cycle dependence associated with the drugs chosen. Rendering an essential nutrient non-metabolizable by chemical modification or blocking a particular pathway alone may not provide a sufficient scope of activity to eradicate all the cancer cell types present. As single agent mechanisms may be reversible and alternative metabolic pathways and nutrients used to escape drug effects. While the concept of drug resistance through protein pumps, excreting drugs from cells many potential escape pathways for metabolic disruptors remain to be identified and characterized.

Using cancer essential nutrients and other agents that can be selectively concentrated in cancer cells as a way to deliver agents capable of causing less escapable or inescapable intracellular disruption, disfunction and cell death may provide a more useful therapeutic benefit to patients either when used alone or as a part of combination protocols including use of current cytotoxic or immune oncology drugs. The present invention provides such a solution. The foundation of achieving cancer selectivity and drug tolerability being the selection of cancer targeting molecules to the receptors and transporters found in the blood, associated with cancer cell plasma membranes or cytoplasm associated with the more unique metabolic requirements and phenotypes of disease. Additional molecules other than nutrients that are cancer targeted and selectively taken up into cancer cells based on electric charge, endocytosis or interaction with plasma membranes may also be used.

### SUMMARY OF THE INVENTION

Briefly, embodiments of the invention described herein include a cancer targeting moiety linked to an oncotic-osmotic molecular crowding agent, described herein generally as a conjugate. When administered to a patient in need of therapy, these conjugates are useful for cancer cell killing by causing an irreversible microfluidic bio-physical cataclysmic change. As most cancer cell types have the necessary receptors and uptake mechanisms, the diversity of cell populations able to be killed is dramatically increased.

In accordance with a first aspect of the invention, there are provided methods of causing mammalian cell death. More specifically, without intent to limit, causing diseased, or disease-causing cell deaths or supporting cell deaths. The methods include contacting a mammalian cell with such a conjugate, or a solution containing a plurality of such polymer conjugates, each conjugate comprising a cellular uptake promoter or as escribed herein a targeting moiety, such as a fatty acid or nutrient or metabolite, or other molecule such as substituted rhodamine or any suitable cation or anion substitution which can be taken up into a cancer cell from exogenous sources, covalently linked to a modulator of colloid oncotic pressure which can be a colloid polymer, such as polyethylene glycol (hereinafter PEG). The contacting of the cell with the conjugate is done under conditions sufficient to allow at least one or a sufficient number of the conjugates to be taken up by the cell to produce the desired effect, i.e., cause cell death. Once cellular uptake and any further distribution to an organelle or membrane compartment has been achieved, the colloid polymer portions of the conjugates increase the intracellular oncotic pressure modifying the osmotic pressure of the cell, causing disruption of organized protein and other functions and cell membrane disruption and death of the mammalian cell. The conjugates remain intact after cellular uptake as a result of the linkages being substantially resistant to intracellular or cleavage in the circulation.

As a result of the methods being capable of causing cell death, the conjugates are useful in the treatment of metastatic and malignant diseases in mammals, especially humans. The methods can be conducted in vitro as well as in vivo on the targeted cells or patient derived biopsies. In the embodiment where the conjugates are used in vitro, they can be employed in diagnostic assays to confirm the presence of malignant cells and how best to treat them. In the embodiment where the methods are used in vivo, the conjugates are administered to a mammal in need of such treatment as part of a pharmaceutically acceptable dosage form such as an intravenous infusion containing one or more desired conjugates, excipients, and formulation agents.

In a preferred embodiment of the invention, pharmacokinetics and dosing schedule allow for sufficient time of exposure to diseased cells for the necessary drug concentration and therapeutic effect, considering levels and saturabilability of receptors and transporters in the blood, and at cancer cell membrane surfaces and in the cytoplasm. In another preferred embodiment of the invention, intravenous infusion rates are such that formation of any drug undesirable aggregates are minimized and an in-line infusion cassette compatible with the drug formulation is included with sufficient plasma pharmacokinetics so as to maximize interaction with the appropriate intended receptors and transporters or other mechanisms of uptake. Too rapid excretion from the body or metabolism to inactive forms could limit achievable concentrations of drug in the target cells.

The methods of the present invention demonstrate that oncotic-osmotic pressure changes can disrupt cell metabolism, such as without intent to limit production of ATP and bio intermediates, as well as cause cell membranes to become leaky or rupturing and leading to cell death. The latter being supported and demonstrated hereinbelow by staining of treated cancer cells (HCT 116) with phalloidin, a dye that detects changes in the cell actin cytoskeleton in response to changes and cell shape and integrity driven by changes in colloid oncotic pressure. Further, as shown in the Examples and corresponding Figures, these changes are dose-dependent and can be seen even at low micromolar concentrations. Surprisingly, substantial differences are observed depending on targeting agent, colloid polymer, e.g., PEG molecular weight (MW), linkage chemistry and concentration in cells. While not wishing to be bound by theory, treatment responsiveness is believed to be dependent on the presence of transporters and receptors that match the targeting agent or cellular uptake promoter conjugated to the colloid polymer, the concentrations achieved and characteristics of the polymers and cellular distribution. One preferred embodiment of the invention includes formulations containing one or more targeting agent polymer conjugate(s) to maximize the diversity of cell kill. Optimal pairing of conjugated targeting agents and PEG composition can be made based on patient derived cell and tissue assays. Differences may be anticipated based on receptor transporter subpopulations and site of intracellular delivery.

In embodiments of the invention, cataclysmic biophysical changes are colloid oncotic-osmotic pressure driven changes in water and ion cellular flux and distribution between organelle compartments, including swelling and membrane lysis. In addition, molecular crowding leads to disruption, destruction dissociation and precipitation of protein organization, chromatin, cell and organelle lipid organization, cytoskeleton membranes, and their integrity. Concurrent or resulting metabolic or signal transduction events secondary to changes in biophysical intracellular effects also occur and contribute to cell death.

In preferred embodiments the conjugates described herein have both oncotic-osmotic pressure and molecular crowding properties contained within the same chemical structure and formulation. Selective targeting to diverse cancer cell types is achieved by suitable sufficiently stable conjugation to an essential nutrient, cofactors, and vitamin ligands recognized by receptors and transporters found in blood and cancer cells and supportive tissues. Without being bound to theory, any molecule selective for delivery to cancer cells can serve as the targeting portion of the conjugate. In preferred embodiments of the invention, uptake mechanisms are unique or highly selective for cancer and their levels correlate with disease aggression improving probability of selective uptake of the killing mechanism.

### BRIEF DESCRIPTION OF THE DRAWINGS

FIGS. 1-6 are graphs illustrating results obtained in Example 1;

FIGS. 7-9 are graphs illustrating results obtained in Example 2;

FIGS. 10A, 10 B, 10C, 10D and 10E are microscopic photographs associated with Example 3;

FIGS. 11A and 11B are microscopic photographs associated with Example 4;

FIGS. 12 and 13 are time lapse photographs corresponding to Example 5;

FIGS. 14A and 14B illustrate dose dependent effects for the C18-Ether-PEG-1000 analogue as described in Example 6;

FIGS. 15A and 15B are photographs illustrating results corresponding to Example 7;

FIGS. 16A, 16B, and 16C are graphs showing the results of cell motility experiments in accordance with Example 8;

FIGS. 17A, 17B, 17C, 17D, 17E and 17F are dry mass graphs corresponding to Example 9;

FIGS. 18A, 18B, and 18C are graphs corresponding to the anti-cancer activity results described in Example 10; and

FIGS. 19A and 19B provide the results of therapeutic assays described in Example 11.

### DETAILED DESCRIPTION OF THE INVENTION

The conjugate structures are illustrated by general Formula (I):

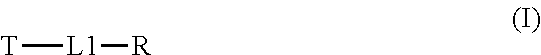

where T is a cellular uptake promoter, a cancer cell essential nutrient or targeting moiety or targeting ligand, delivered to cancer cells by plasma or cancer cell membrane receptors and transporters. These receptors are uniquely, over expressed, or dysregulated in cancer and present in-patient blood and cells. Alternatively, where passive or facilitated diffusion or endocytosis may be a characteristic of diseased or disease-causing cells and the ligand chosen to promote uptake. The targeting ligands include cancer essential nutrients such as glucose, fatty acids, lipids, amino acids such as glutamine, arginine, asparagine, vitamins, and cofactors with anionic or cationic features and their analogues. Alternatives include, for example, without limitation, compounds confer a charge such as substituted rhodamine or other anions, or cations, e.g., phosphonium, dequalinium, nitronium, etc.

In a preferred embodiment, the targeting ligand is a short, medium or long chain fatty acid, saturated or unsaturated, cis or trans isomers of 4-20 carbon length with alcohol or other substitutions as directed by SAR biological assays and physical properties preserving cancer uptake features and benefits according to the disease intended to be treated, with long chain fatty acids and their derivatives being most preferred.

L1 represents a bonding chemistry or a linker joining the targeting moiety (T) to (R) providing chemical stability, solubility, enhanced circulating lifetime and resistance to metabolism. Preferred linkages include but are not limited to, amides, ethers, ureas, carbamates, vinylogous amides and squaramides, with ether, vinylogous amide and squaramide linkages being more preferred.

R represents the modulator of colloid oncotic pressure (COP) and molecular crowding. R may be selected from among dextrans, saccharides, synthetic polymers, block co-polymers and propylene glycol, with polyethylene glycol (PEG) being most preferred. The PEG can be a mono or poly dispersed polymer of molecular weight average from about 750 to about 20,000, with preferred molecular weights of between about 1000 and about 5000.

### Features of the Invention Leading to Cancer Cell Death

### Oncotic Pressure

Oncotic pressure is an effect where proteins, particles, and colloidal polymers pull water and ions into that compartment where the proteins, particles, and colloidal polymers are the most concentrated. The pulling force of osmosis that tries to equalize the amount of water on both sides of the compartment membrane is called oncotic pressure. Changes in the intracellular oncotic pressure of cancer cells modulates structure, function, motility and invasiveness and survival of tumors and cancer cells. (Kao, Y C., Jheng, J R., Pan, H J. et al. Elevated hydrostatic pressure enhances the motility and enlarges the size of the lung cancer cells through aquaporin upregulation mediated by caveolin-1 and ERK1/2 signaling. Oncogene 36, 863-874 (2017)). We describe chemical structures that selectively target oncotic pressure modulating agents to diverse cancer cell types for the treatment of cancer patients.

### Molecular Crowding

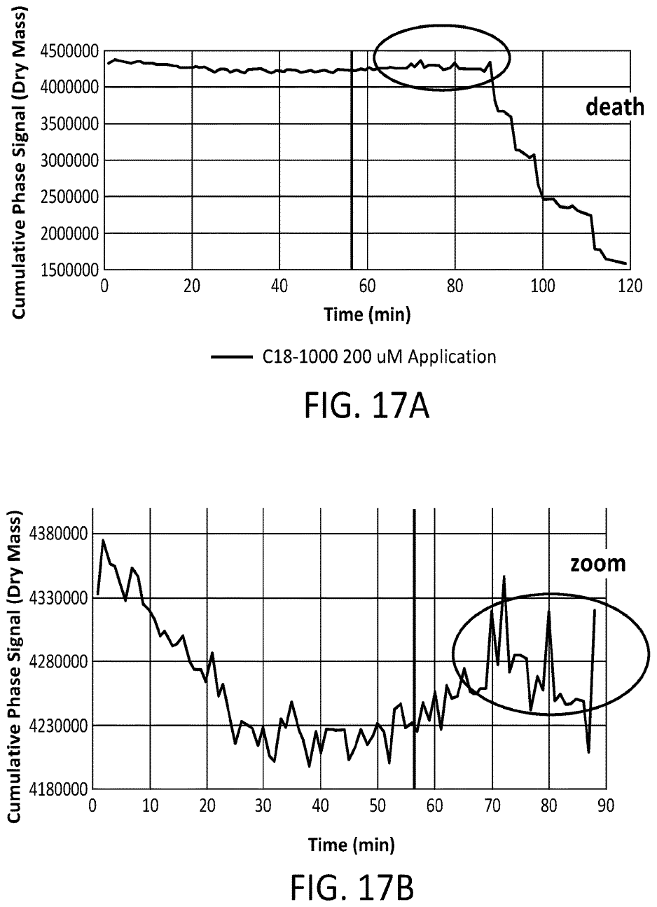

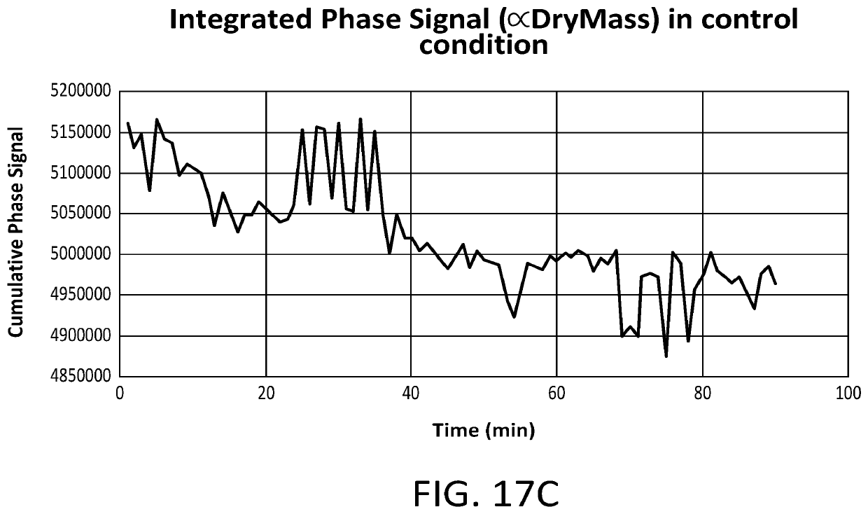

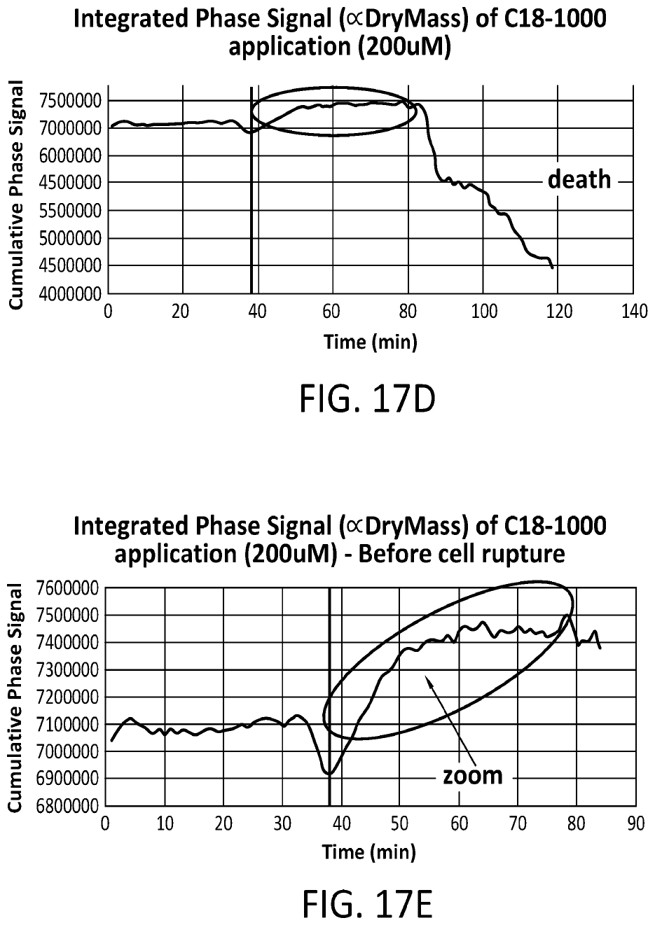

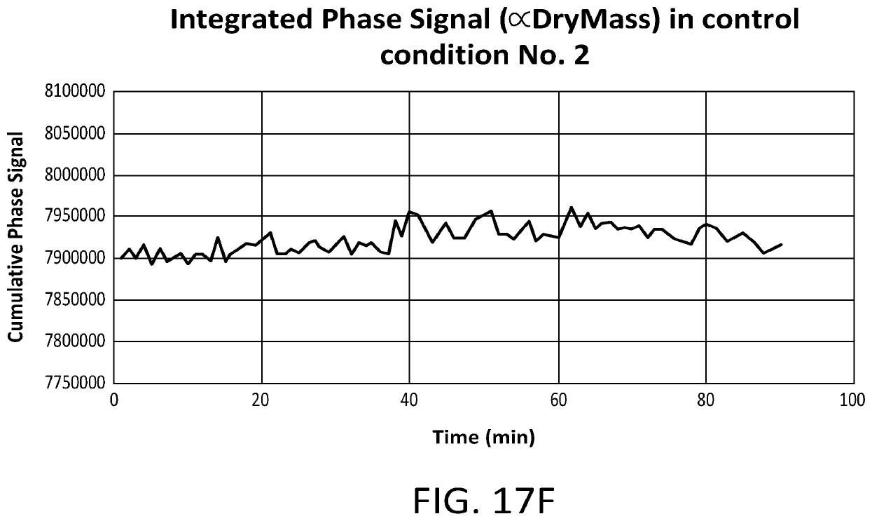

Molecular crowding occurs when high concentrations of macromolecules reduce the volume of solvent available for other molecules in the solution, effecting the properties of cellular water. (Chemical Reviews 2024. DOI: 10.1021/acs.chemrev. 3c00615., Anal. Chem. 2019, 91, 4, 2586-2590 Publication Date: Jan. 9, 2019. With less water available, crowding promotes the formation of a biomolecular condensate and colloidal phase separation. Studies of molecular crowding conditions revealed changes in protein structure, folding, shape, conformational stability, binding of small molecules, enzymatic activity, protein-protein interactions, protein-nucleic acid interactions, and pathological aggregation (Colloid osmotic parameterization and measurement of subcellular crowding T. J. Mitchison, Doug Kellogg, Monitoring Editor 14 January). We have used Holographic light microscopy to measure the dry mass, molecular crowding, membrane rupture and death in single cells and clusters on exposure to drug. An increase in dry mass on addition of drug to the cells reflects uptake of the conjugate into the intracellular space. At a sufficient concentration of drug and change in intracellular microfluidics, cells offload their intracellular contents as part of the cell death process, see Example 9, infra and FIG. 17A. The method has been used to evaluate a dose response relationship and the characteristic response patterns of specific cell types to selected polyethylene glycol fatty acid conjugated structures. Polyethylene glycol uniquely acts as both an oncotic pressure and a molecular crowding agent and is the most preferred polymer in embodiments of the invention.

### Molecular Description of the Invention

There is extensive literature available that teaches that the polymer (PEG) is taken up into cells in small amounts by passive diffusion or endocytosis depending on cell types, molecular weights, geometry, concentration, solvents, and salts. In general PEG is considered for use in the prolongation of the circulating life of small molecule and protein drugs and as carriers for prodrugs (Wang T, Guo Y, He Y, Ren T, Yin L, Fawcett J P, Gu J, Sun H. Impact of molecular weight on the mechanism of cellular uptake of polyethylene glycols (PEGs) with reference to P-glycoprotein. Acta Pharm Sin B. 2020 October; 10(10):2002-2009. More specifically, polymers such as PEG with molecular weights from 750 to 40,000 (~17 to 910 repeating ethylene glycol units) with diverse geometry and chemistry have been frequently used conjugated to proteins and small molecule drugs and phospholipids to limit cellular uptake, prolong circulatory lifetimes, limit immunogenicity, limit drug metabolism and distribution and act as prodrugs and components in liposomal and other sustained drug delivery systems to control rate of release of active drugs. PEG with a molecular weight of less than 350 (<8 repeating ethylene glycol units) may diffuse into cells but are generally regarded as toxic. A surprising and remarkable enabling aspect of the disclosed invention is the discovery that PEG conjugates to certain ligands can be selectively delivered and taken up by cancer cells. Allowing a stable-linked PEG to a cancer selective ligand receptor or uptake promoter to modify colloid oncotic-osmotic pressure and molecular crowding producing a physiochemical alteration of the cells leading to death. Thus, contrary to this art, we have discovered that intracellular microfluidics and molecular crowding of cancer cells can be modulated by PEGs as a treatment for cancer.

### The Colloid Polymer Portion (R)

The modulator of colloid oncotic pressure, e.g., polymer, portion of the conjugates used in the methods of the invention, designated in formula (I) as (R) or R² as set forth in Formulae (II)-(VI) below, is preferably a polymer associated with higher colloid oncotic pressure and molecular crowding, especially in the intracellular environment. Without intent to limit such polymers useful in the inventive methods, include polyalkylene oxides such as PEG. The polymers preferably have a weight average molecular weight of at least about 350. In further aspects, the polymer such as PEG will have a weight average molecular weight of from about 750 to about 20,000, with weight average molecular weights of from about 1,000 to about 5,000 being preferred. The polymers may be linear or branched, star- or multi-armed, and activated for covalent attachment of the cellular uptake promoter on one or more ends. Molecular weight and end group blockage e.g., small alkoxy like methoxy PEG must be sufficiently stable to prevent PEG, or alternate chain degradation. It is preferred that the PEG have a polydispersity of about 5%, or less than 2% most preferred, and meet all regulatory requirements for use in medical administration. Conjugates of absolute molecular weights and little or no poly dispersity can also be prepared (Milla, et al., “PEGylation of Proteins and Liposomes: a Powerful and Flexible Strategy to Improve the Drug Delivery”, Current Drug Metabolism, 13, 1, (2012): 105-119) (Wu, et al., “Drug Development through Modification of Small Molecular Drugs with Monodispersed Poly(ethylene glycol)s”, Org. Process Res. Dev., 24, 8, (2020): 1364-1372).

Such activated polymers, especially activated PEGs and methoxy capped PEGs (mPEGs), are commercially available from multiple sources and the conjugation chemistry for attachment of the PEG to a target is within the level of skill of the artisan without undue experimentation. See, for example Harris, J. M. et al. Effect of Pegylation on Pharmaceuticals, Nature Reviews Drug Discovery 2, 214-221 (2003), the disclosure of which is incorporated herein by reference. In many aspects, the PEG is a linear PEG and the cellular uptake promoter or targeting moiety (T) is covalently linked to one terminal of the PEG and the distal end of the PEG is non-reactive. Alternatively, the PEG can be bi-functional or multifunctional and a cellular uptake promoter or targeting moiety is covalently linked to each terminal of the PEG.

In many embodiments, the conjugates of Formula (I) are formed by reacting an activated form of a polymer, i.e. a polymer having a linker-forming group on a terminal end, with the targeting molecule, such as a fatty acid, under conditions known to those of ordinary skill, to facilitate linkage of the polymer to the targeting moiety and form the conjugate.

Although polyethylene glycol is a preferred polymer, polypropylene glycol, and hybrid copolymers of any geometry and molecular weight suitable for the desired effect can be used. In alternative aspects of the invention, the polymer can be selected as one of the following examples without intent to limit, a polymer associated with higher colloid oncotic pressure such as polyvinylpyrrolidone, polyvinyl methyl ether, poly hydroxy propyl methyl acrylamide, poly hydroxy propyl methacrylate, poly hydroxyethyl acrylate, poly methyl acrylamide, poly dimethyl acrylamide, poly methyl oxazoline, polyethyloxazoline, polyhydroxyethyloxazoline, poly hydroxypropyoxazoline, polyaspartamide, dextrans, celluloses, various block copolymers containing PEG. In still further aspects, the polymer may also be a polymeric, blocked copolymeric linear or branched chained dendritic nanoparticle of 0.05-1 micron with 0.1-0.2 micron most preferred. In alternative aspects, the polymer can be colloidal particles such as but not limited to nanoparticles or emulsions which are capable of being covalently bound to an essential nutrient or cellular uptake promotor as described herein. In additional embodiments of the invention structures may be such that, in addition to modification of colloid osmotic pressure, surfactant activity may be present but not required. In all cases, sufficient concentration of the oncotic-osmotic pressure, molecular crowding modifier must be achieved so as to raise sufficient osmotic pressure or molecular crowding, or otherwise disrupt metabolism or membrane integrity to achieve the desired result of cell death.

### Cellular Uptake Promotors (T)

The second principal component of the conjugates corresponding to Formula (I) and used in the methods of the invention is the cellular uptake promotor or targeting moiety, designated herein as (T) and T¹ as set forth below in Formulae (II)-(VI). These terms cellular uptake promotor or targeting moiety are used interchangeably throughout the description. In many aspects, suitable cellular uptake promotors are cancer essential nutrients, cations and anions which are capable of being recognized and attached to selectively expressed receptors and transporters for the uptake and delivery to the appropriate cancer cells. As a result of being covalently attached to the colloid polymer, the conjugates are carried selectively to cells and organelles where the colloid polymers alter oncotic-osmotic pressure and molecular crowding to induce cancer cell death.

Broadly speaking, the cellular uptake promotor or targeting moiety portion of the conjugate used in the methods of the invention can be any nutrient, peptide, antibody, or congeners or any ligand alone or in a desirable combination shown to be selectively taken up by cancer cells. Without intent to limit additional structures promoting uptake into cancer cells and organelles based on charge and the characteristic differences in membrane potential of the plasma and mitochondrial membrane may be used e.g., phosphonium and nitronium. The only limitation is that the cellular uptake promotor must be capable of forming a stable linkage, i.e., via L1, to a polymer, i.e. (R), while maintaining an affinity for plasma and cell receptors or transporters which allow uptake by targeted cells. The targeting agent can therefore be selective in delivering the colloid polymer, e.g., PEG or, generally, (R) to a desired organelle or intracellular location.

In one embodiment of the invention, fatty acid, derivatives, and congeners required or preferred as nutrients or recognized by the selective receptors and transporters corresponding to (T) of Formula (I) are conjugated, i.e., linked via L1, to a hydrophilic or amphiphilic polymer or surfactant (R). Such polymers capable of having the desired effect on oncotic-osmotic pressure, being recognized by the receptors and transporters, and not inhibiting cell uptake, for example, and without limitation, fatty acid and lipid binding proteins and transporters. The conjugate thus acts as a cancer selective metabolic disruptor, an oncotic osmotic pressure modulator and molecular crowding agent. Changes in osmotic pressure, thereby producing swelling, leakiness or rupturing of cancer cell and compartmental membranes leading to cancer cell death, stasis, or both. More specifically, fatty acids, derivatives, and congeners thereof conjugated to a hydrophilic polymer such as but not limited to polyethylene glycol.

As mentioned, conjugated alkane chains like fatty acids are one preferred cellular uptake promoter for inclusion in the conjugates. The alkane chain and fatty acids may be in saturated, unsaturated, cis or trans, glyceride form and any derivatives thereof including enantiomers. In further aspects, the cellular uptake promotor can be alkane chains, fatty acid analogs, sugars, amino acids, or lipids including combination in a hetero functional linear or branched chain polymer. In those cases where an alkane chain or fatty acid is used, non-limiting suitable examples are of the formula CH₃(CH₂)ₙCOOH, CH₃(CH₂)ₙCONH₂, CH₃(CH₂)ₙCH₂OH, or CH₃(CH₂)ₙCH₂NH₂, where n is a positive integer, of odd or even number such as from about 2 to about 20, or from about 6 to about 18 as preferred. The alkane chain may be saturated or unsaturated or polyunsaturated. After formation of the conjugate, the fatty acid is linked to (R) as CH₃(CH₂)ₙCOO-L1-R, CH₃(CH₂)ₙCH₂O-L1-R, CH₃(CH₂)ₙCONH-L1-R, CH₃(CH₂)ₙCH₂NH-L1-R.

For example, and without intent to limit, some useful fatty acids/alcohols/amines include capric, lauric, myristic, palmitic, palmitoleic (D9-Z), (2, 3, 4, 5, 6, 7 or 8-oxodecanoic, arachidic, behenic, octanoic acid, stearic, oleic, linoleic, alpha-linolenic, palmitic, lauric acid/alcohol/amine, derivatives, and congeners, with stearic acid/alcohol/amine and lauric acid/alcohol/amine being preferred in some aspects of the invention.

### Conjugate Linker (L1)

The polymer portion and cellular uptake promotor or targeting moiety are preferably combined with a linkage of suitable stability to optimize desired features and benefits. While polymer conjugation chemistry is known to include the use of activated polymers which facilitate the conjugation to a target using various types of linkages, such as an ester, an amide, secondary amide, vinyl, urethane (carbamate), ether, thioether, disulfide, urea, vinylogous amide, deltamide (substituted cycloprop-2-ene-1-one), squarates and squaramides (substituted cyclobut-3-ene-1,2-dione) and, croconamides (substituted cyclopent-4-ene-1,2,3-trione) [(1) Zwicker, V. E.; Yuen, K. K. Y.; Smith, D. G.; Ho, J.; Qin, L.; Turner, P.; Jolliffe, K. A. Deltamides and Croconamides: Expanding the Range of Dual H-bond Donors for Selective Anion Recognition. Chemistry—A European Journal 2018, 24 (5), 1140-1150. DOI: https://doi.org/10.1002/chem.201704388]. Preferred linkages for use herein are those which resist extracellular and intracellular degradation and thus keep the polymer linked to the essential nutrient or targeting agent in the cell. In some preferred aspects of the invention, the linkage is an ether, an amide or a cyclic vinylogous amide like squaramide which may contain a cationic or anionic linker.

For purposes of illustration, an activated form of methoxy-capped PEG (mPEG), such as PEG-1000-NH₂ or other forms of activated PEG's can be reacted with stearic acid or other fatty acids or targeting moieties to form conjugates under conditions as set forth in Harris, supra, in addition to a pharmaceutically acceptable spacer with desirable characteristics to form the bond linking the polymer, without intent to limit, maybe an acyl, alkyl, alkoxy, alkenyl, alkenyloxy, alkynyl, aryl, heteroaryl, alkyl sulfide, alkyl sulfide, imidoyl, hemiacetal unsubstituted or substituted; wherein the substitutions may be one or more of hydrogen, alkyl, alkenyl, alkynyl, cycloalkyl, aryl, alkylaryl, heteroaryl, or heterocyclyl, aromatic any of which can be substituted or unsubstituted. In addition, mPEG functionalized with a carboxylic acid (PEG-OCH₂COOH, PEG-OCH₂CH₂COOH or PEG-OCH₂CH₂CH₂COOH) may be coupled C₁₂-C₂₀ chains (saturated or not) terminating with an amine group (1° or 2°). Another illustrative example includes first reacting diethyl squarate with a fatty alcohol and thereafter reacting the resultant intermediate with PEG-NH₂. Further considerations include a metal chelate, F substitutions including a metal complex. Stabilized double or triple bonds, dienes, and stable covalent ionic bonds without limitations. Illustrative conjugates with some preferable linkages are provided below as Formulae (II)-(VI):

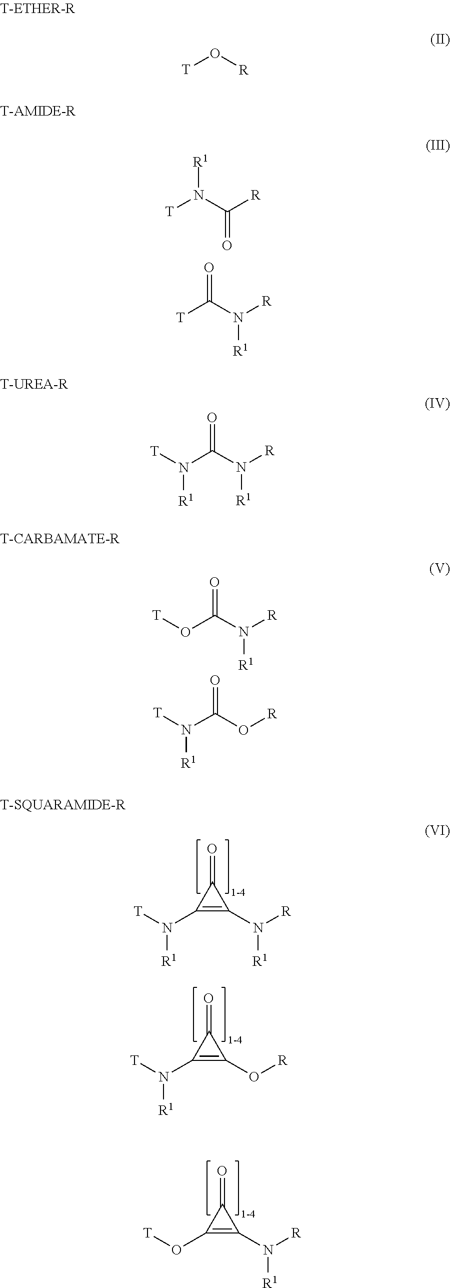

where T and R are as described above and R¹=H, C₁-C₆ alkyl, C₃-C₁₂ cycloalkyl, C₃-C₁₂ heterocycloalkyl with 1-3 heteroatoms, or OH.

While applicants are not bound by theory, it is believed that due to the conjugation, the fatty acid or targeting agent is rendered non-cleavable (non-usable) i.e., unable to support required cancer metabolism, by stabilization of bonds but it is still being absorbed into the cells. This is typically modified by the enzymes reconfigured in structure and function from healthy cells, so as to meet cancer metabolic needs. In preferred embodiments of the invention essential nutrients such as fatty acids, derivatives and congeners are rendered non-usable and disruptive by conjugation to a polymer, nanoparticles, or emulsion. In the most preferred embodiments of the invention, the modified essential nutrients are preferably recognized for selective cell uptake and delivery to cellular compartments where essential metabolism occurs, thereby engaging as a disruptor of essential metabolic pathways while also delivering the conjugated polymers, nanoparticles, or emulsions. The polymers, nanoparticles, or emulsions acting as modulators of colloid oncotic-osmotic pressure and molecular crowding. Further disrupting metabolism in the targeted cell and desired cell compartments by inducing swelling, depolarization of membranes, or otherwise rendering such membranes leaky or rupturing, to activate cell death pathways. Depending on cell type the principal contributor to cell death may be the inability to metabolize essential or preferred nutrients or modification of oncotic-osmotic pressure and surfactant activity of the conjugates or any manner of combination of these activities.

In some embodiments of the invention, the addition of oncotic-osmotic pressure and molecular crowding characteristics as part of the metabolic disruptor structure. i.e., conjugate, will, in a dose responsive manner, broaden the scope of activity against diverse cancer cell types. This will lessen the requirement for use of traditional cytotoxic anticancer drugs that are not well tolerated by patients in some cases. It is of great importance that cells responsible for metastases be eliminated as ultimately prevention of metastatic disease is likely to result in the greatest increase in overall survival and quality of life for solid tumor patients.

The therapeutic effect of the conjugate can be further enhanced by the polymer R or cellular uptake promotor T having at least one atom which is deuterated or radioactive. It should be kept in mind that the conjugates used in the methods of the invention are capable of at least one of (i) disrupting essential metabolic pathways, (ii) disrupting signal transduction pathways and (iii) activating one or more cell death pathways.

In some embodiments of the invention, these agents act according to one or more of the following manners in the desired cells and compartments, such as but not limited to (i) increasing oncotic-osmotic pressure leading to swelling (ii) acting with molecular crowding activities such as changes in microfluidics, chromatin and protein structure and function. ((iii) starving cells of essential or preferred nutrients, thereby activating apoptotic, necrotic and autophagic pathways and cell death. Sufficient levels of these therapeutic agents are required according to the desired cancer cell types and disease as present in a mammalian patient.

### Representative Structures of the Invention

Some conjugates corresponding to Formula (I) and which are useful in carrying out the methods of the invention include:

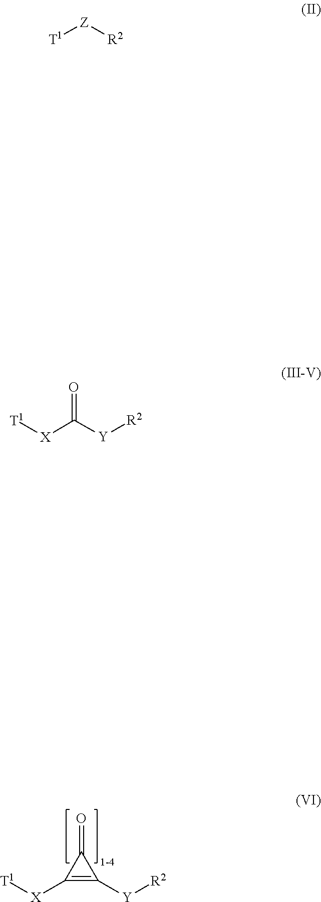

wherein T¹=C₈-C₂₂ alkane, C₈-C₂₂ unsaturated alkane (1-5 unsaturations, each unsaturation independently Z or E steroisomer);
X and Y can be independently CR¹R³, O, NR¹, or S;
R¹=H, C₁-C₆ alkyl, C₃-C₁₂ cycloalkyl, C₃-C₁₂ heterocycloalkyl with 1-3 heteroatoms, or OH;
R³=H, C₁-C₆ alkyl, C₃-C₁₂ cycloalkyl, or C₃-C₁₂ heterocycloalkyl with 1-3 heteroatoms;
R¹ and R³ may be attached together to form a 3-, 4-, 5-, 6-, 7- or 8-membered ring with 1-3 heteroatoms;
Z=O, S, SO², SONH, Se, NR¹, (NR¹R³)⁺;
R²=

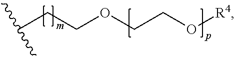

m is an integer from 1 to 8, p is an integer from 15 to 120, or R²=branched PEG chain MW<1500 or other polymer; and
R⁴=H, 1-7 atom alkyl, branched alkyl, cycloalkyl or heterocycloalkyl.

### Some Preferred Conjugates are:

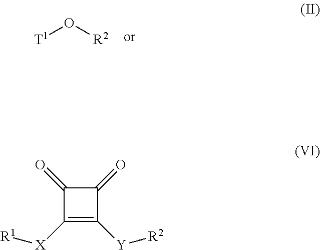

T¹=C₁₀-C₂₀ alkane, C₁₀-C₂₀ unsaturated alkane (1-2 unsaturation, each independently Z or E)
X and Y can be independently O, NR¹
R¹=H, C₁-C₆ alkyl, C₃-C₁₂ cycloalkyl, C₃-C₁₂ heterocycloalkyl with 1-3 heteroatoms, or OH;
R³=H, C₁-C₆ alkyl, C₃-C₁₂ cycloalkyl, or C₃-C₁₂ heterocycloalkyl with 1-3 heteroatoms;
R²=

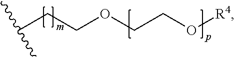

m is an integer from 1 to 8; p is an integer from 20 to 25, or
R²=branched PEG chain MW<1500 or other polymer.

### Additional Preferred Conjugates:

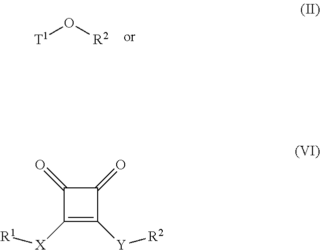

T¹=C₁₀-C₂₀ alkane, C₁₀-C₂₀ unsaturated alkane (1-2 unsaturation, each independently Z or E)
X and Y can be independently O, NR¹
R¹=H, C₁-C₆ alkyl, C₃-C₁₂ cycloalkyl, C₃-C₁₂ heterocycloalkyl with 1-3 heteroatoms, or OH;
R²=

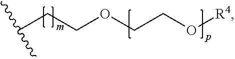

m is an integer from 1- to 8, p is an integer from 19 to 22,
or R²=branched PEG chain MW<1500 or other polymer.

### Representative Ethers

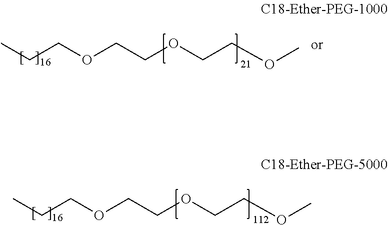

### Representative Amides

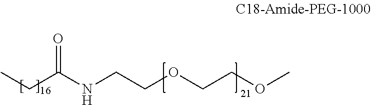

### Representative Squaramides

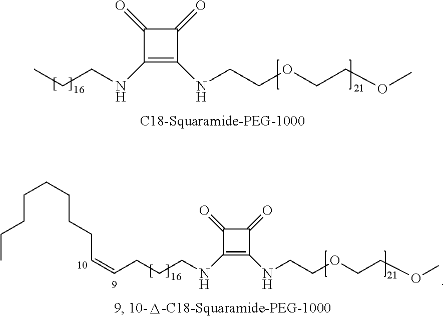

In each of the above representative conjugate examples, it will be understood by those of ordinary skill C18 fatty acid can be replaced with any group corresponding to T or T¹ and that the number of repeating units for the PEG can be any number corresponding to the variable p, e.g., 15-120.

### PK/PD

For a drug to be maximally effective, it is essential that the circulating lifetime be sufficient so that the drug has the opportunity to accumulate in cells and tissues to a sufficient quantity to meet the required concentrations for activity. Further the duration of exposure needs to match the kinetics or the dynamics of drug target events that lead to the desired outcome. In this case cancer cell death. One way in which pharmacokinetics can be made to support pharmacodynamic requirements is by controlling the level and rate of drug infusion, usually by an intravenous route of administration. With a sufficient understanding of bioavailability to the circulation by other routes of administration this requirement can be attempted to be met. PEGylation has emerged as a methodology in its own right for conjugation to drugs with the specific intention to enhance circulating lifetime of desired drugs. In contrast to the most frequent use of PEG conjugation for elongation of circulating life, embodiments of the invention disclosed herein is the uptake of the polyethylene glycol into tumors and cells that is the defining factor in drug effects. Therefore, the circulating lifetime of the preferred embodiments must be such that sufficient levels of polyethylene glycol are delivered within the cancer cells to promote the changes in oncotic, osmotic, and molecular crowding properties necessary to trigger cell death. This aspect of the invention is taught against by the prevailing polyethylene glycol conjugation art where the polymer is intended to limit cellular uptake and filtration through tissues so that drug availability is enhanced by increased time in the circulation. For drugs that interact with cell membrane receptors such as alpha interferon the effects on circulating life and patient benefit of PEG conjugation are substantial. Where a drug is required to be taken up into cells while polyethylene glycol conjugation can increase circulating life there is diminished levels of cellular uptake creating the need for cleavable conjugation and the establishment of varying degrees of sophisticated prodrug approaches where PEG is removed, and the drug is now free to be taken up into cells. In the most preferred embodiments of the invention described herein the rate of cellular uptake and the triggering of changes in the biophysical properties of cells that trigger death are key factors in selecting the polyethylene glycol molecular weight and structure and the conjugation chemistry. In the latter case this stability of the conjugates must be such that there is little or no breakdown of the conjugate structure without loss of interaction with receptors and transporters for the selected fatty acids. This further highlights the need for the selection of an appropriate fatty acid or fatty acid analog to maximize the rate and level of cellular uptake. Substantial differences in fatty acid carbon chain length have been noted with dramatic differences observed in effects on various cancer cell lines tested PEG is considered chemically inert and the half-life of the molecule on its own increases from 18 minutes to one day as the molecular weight increases from 6000 to 190,000.

### Methods of Treatment

The methods of the treatment are generally directed to causing cancer cell death i.e., malignant, or metastatic cells associated with disease. The conjugates of the present invention are particularly suited for treatment of primary tumors, metastatic lesions, leukemias, etc. and cancer stem or metastasizing cells in the circulation of mammalian patients. Without wishing to be limited, the conjugates can be used to treat cancers or malignancies involving prostate, breast, pancreatic, lung, renal, ovarian, skin, cervical, glioblastoma, brain, osteosarcoma, head and neck, colorectal, testicular, thyroid, leukemia, myeloma, without limitation other than characterized as dependent on the targeting agent selected and the presence of the associated with the appropriate receptors and transporters.

The in vivo treatment methods wherein the cell is part of a mammal can include administering pharmaceutically acceptable dosage form such as a solution containing a plurality of polymer conjugates to the mammal in need of such treatment. In many embodiments, the conjugates are part of a pharmaceutically acceptable dosage form which is a parenteral dosage form such as an intravenous solution. The amount of the polymer conjugates administered to the mammal is generally an amount which is effective to cause a therapeutic result. The dosage will depend on the conjugate components and the condition being treated. The amounts administered can be from about 0.01 to about 1000 mg/kg given in one or more doses, based upon clinical experience.

The methods may also be expanded to include administering the conjugates in combination with other pharmaceutically active agents or drugs, metabolic disrupters, cytotoxins, and immunotherapies, etc. for maximal benefit. The conjugates may be combined with the auxiliary agents in the same dosage form or administered separately as part of a combination therapy wherein the conjugates and pharmaceutically active agents are given separately according to predetermined therapeutic regimens. A therapeutically effective amount of at least one of any of the aforementioned conjugates may be administered to a subject for the treatment of a disease, or symptoms thereof, in mammalian patients by parenteral and non-parenteral methods.

Generally, the compounds/conjugates of the present invention are delivered in a manner sufficient to administer to the patient an amount effective to deliver the agent to its intended molecular target. In some further embodiments, the dosage amount may range from about 200 mg/m² to about 3000 mg/m². The dosage amount may be administered in a single dose or in the form of individual divided doses, such as from one to four or more times per day. In the event that the response in a subject is insufficient at a certain dose, even higher doses (or effective higher doses by a different, more localized delivery route) may be employed to the extent of patient tolerance.

For purposes of the present invention, it will be understood by those of ordinary skill in the art that the conditions under which the mammalian cells are contacted by the conjugates of Formula (I) include physiologic conditions such as when a patient is administered a conjugate as a part of a therapeutic protocol in which a conjugate is delivered to the patient in need thereof such as by any recognized route of administration, e.g. parenteral (intravenous) or oral. If in vitro, under conditions recognized as generally accepted laboratory conditions.

### Formulations Containing Colloid Polymer Conjugates

The pharmaceutical formulations of the present invention can be prepared according to conventional formulation techniques and may take any pharmaceutical form recognizable to the skilled artisan as being suitable. Suitable pharmaceutical forms include solid, semisolid, liquid, or lyophilized formulations, such as tablets, powders, capsules, suppositories, suspensions, liposomes, emulsions, nano emulsions, aerosols, sprays, gels, lotions, creams, ointments, and the like differing from specific examples named herein. If such a formulation is desired, other additives well-known in the art may be included to impart the desired consistency and other properties to the formulation. For example, a stock solution of the compounds can be prepared according to conventional techniques and then diluted as desired by a pharmaceutically acceptable diluent to form a liquid preparation such as a sterile parenteral solution. For example, without intent to limit 100 mg/mL in water or PBS.

The pharmaceutical formulation of the present invention may be administered using any mode of administration both that is medically acceptable and that produces effective levels of the agent without causing clinically unacceptable adverse effects. The pharmaceutical formulations of the present invention may be contained in any suitable vessel, such as a vial or ampoule, and suitable for multiple dosing routes including inhalational, oral, topical, transdermal, nasal, ocular, pulmonary, rectal, transmucosal, intravenous, intramuscular, intradermal, subcutaneous, intraperitoneal, intrathoracic, intrapleural, intrauterine, intra-tumoral, or infusion methodologies or administration, without limitation. Those skilled in the art will recognize that the mode of administering the analog or derivative of the present invention depends on the type of disease or symptom to be treated. Likewise, those skilled in the art will also recognize that particular pharmaceutically acceptable carriers or excipients will vary from pharmaceutical formulations suitable for one administration mode to those suitable for another administration mode.

Formulations containing more than one species of conjugate are included in further aspects of the invention. For example, a therapeutically beneficial formulation will include a plurality of a first conjugate wherein a first fatty acid is conjugated to a first mPEG-1000 and a plurality of a second conjugate comprising a multi-arm PEG-1200 conjugated to multiple fatty acids which are different from the first fatty acid. Suitable formulations and treatment methods may further include other therapeutic compounds or biologics for the purpose of enhancing therapy. In an additional embodiment of the invention a selected conjugate is further conjugated to a higher molecular weight PEG by a cleavable bond serving as a prodrug for sustained release of the lower molecular weight PEG conjugate.

The conjugates designed with a cellular uptake promotor having specificity for a specific cancer cell uptake receptor, conjugated to a PEG can also be used in diagnostic in vitro procedures. The conjugates are allowed to contact a patient sample and the amount of cell death occurring over a period of time is observed. Without intent to limit such receptors may be present in the circulation or associated with cancer cell membranes and cytoplasm such as fatty acid binding proteins, fatty acid transport proteins CD 36 and other nutrient receptor and transporters associated with a selected targeting agent. Such as glucose transporters and receptors-transporters for amino acids.

Another embodiment of the invention is where the polymer maybe branched chain with one or more hetero or homo targeting ligand at each polymer termini or with a capped end for example without intent to limit a methoxy group. Further, a prodrug construct where the active conjugate is linked via a cleavable bond to a polymer of 5000-40,0000 molecular weight or incorporated into a micelle, nano emulsion, or particle.

### EXAMPLES

### Example 1

In this example, comparative testing was done using HCT-116 cell spheroids to show that the glycerol esters of fatty acids (Lauric acid) were superior in activity compared to non-conjugated forms of the fatty acid, or PEG or the vehicle alone. Monolaurin was also similarly compared as a non-pegylated Lauric acid control.

HCT-116 cells were cultured according to ATCC guidelines and spheroids containing the cells were incubated in plate maps. To compare the effects of the C₁₂-PEG-1000-Ether) and C₁₈-amide-PEG-1000 conjugates on the cell proliferation of the HCT-116 spheroids, the spheroids were stained with DAPI, Alexa Fluor 488, Phalloidin, Mito-Health, Mito-Tracker and Viability. After 72 hours of incubation, the images were taken at 10× with a 25-step z-stack in 10 μM slices on a Molecular Devices imager.

On the day of dosing, C12 (C12=C12-Ether-PEG-1000), C-18 (C18=C18-amide-PEG-1000) and monolaurin were reconstituted in DMSO to make stock solutions of 60 mM. Lauric Acid and Polyethylene Glycol were also reconstituted in DMSO to make stock solutions of 120 mM. C-12, C-18 and monolaurin stock solutions were then serially diluted at half-log steps in DMSO and then diluted 100× in maintenance media. Lauric Acid and monomethoxy polyethylene glycol MW 1000 (PEG) were serially diluted in DMSO and then diluted 200× in maintenance media. Dosing done according to manufacturer's protocol.

DMSO concentrations were kept consistent across all wells at 0.5%. Following dosing, Plate 1 & 2 were incubated in a 37° C., 5% CO2 incubator for 72 hours. Plate 1 was removed from incubation between 60 and 180 min timepoints for imaging. Well A05 in Plate 1 was imaged in bright field at 10× while incubated in the imager at 37° C. Images were taken at 5-minute intervals for 120 mins. Following imaging, Plate 1 was immediately placed back into 37° C., 5% C₀₂ incubator.

On day 5, staining solutions were prepared. 15.75 μL of Mito-Health Stain was added to 8,984.25 μL of complete media. 220 μL of Mito-Tracker Deep Red Stain was added to 21,780 μL of basal McCoy's 5A Medium. 44 μL of Viability stain was added to 21,956 μL 1×PBS. At conclusion of 72 hours, plates were removed from 37° C., 5% CO2 incubation. Plates were immediately observed, and all wells were confirmed to have maintained presence of spheroid.

From each well 100 μL was removed and 40 μL of Mitohealth Stain solution was added to all wells and placed in 37° C., 5% CO2 Incubator for 30 mins. Following 30 min incubation, plates were removed and 60 μL of basal McCoy's 5A Medium was added to all wells. 2 half-exchanges were performed in all wells with basal McCoy's 5A Medium.

Following exchanges, 100 μL was removed from all wells and 100 μL of Mito-Tracker Deep Red Stain solution was added to all wells. Plates were placed into 37° C., 5% C₀₂ incubator for 30 mins. Following 30 min incubation, plates were removed, and 2 half-exchanges were performed with 1×PBS. PBS was allowed to incubate in wells for 5 mins after each exchange. Following exchanges, 100 μL was removed from all wells and μL of Viability Stain solution was added to all wells. Plates were left to incubate at room temp for 30 mins.

Following incubation at room temperature, 2 half-exchanges were performed with 1×PBS. PBS was allowed to incubate in wells for 5 mins after each exchange. Following exchanges, 100 μL 10% neutral formalin buffer was added to all wells and was allowed to incubate at room temp for 30 mins. Following 30 min incubation, plates were removed, and 3 half-exchanges were performed with 1×PBS. PBS was allowed to incubate in wells for 5 mins after each exchange.

Following final exchange post-fixation, plates were wrapped in parafilm and stored at 4° C. overnight. On day 6, plates were removed from 4° C. storage and 1:100 Phalloidin and 1:200 stock of DAPI in 1×PBS was made. 100 μL was removed from all wells, and 100 μL of PBS containing 0.2% Triton X-100 permeabilization buffer was added. Spheroids were incubated in permeabilization buffer at room temp for 5 mins. Following permeabilization, 2 half-exchanges were performed with 1×PBS. PBS was allowed to incubate in wells for 5 mins after each exchange. 100 μL was removed from all wells and 100 μL of Phalloidin/DAPI stain was added to all wells and incubated at room temp for 30 mins on orbital shaker.

Following 30 min incubation, 2 half-exchanges were performed with 1×PBS. PBS was allowed to incubate in wells for 10 mins after each exchange. Following exchanges, spheroids were dehydrated in a methanol gradient then cleared with Visikol HISTO-M™ prior to imaging.

Spheroids were imaged at 10× with 10 μm z-steps using a Molecular Devices ImageXpress Micro Confocal High-Content imaging platform. The scan laser/dichroic settings were tuned for 405 (DAPI), 488 (Phalloidin), 555 (MitoHealth), 640 (MitoTracker), and 730 (Viability) nm excitation wavelengths. Images were acquired and saved as 16-bit 2048×2048 TIF files for further processing.

Clearing the spheroid with Visikol HISTO-M™ allows for clear images throughout the spheroid volume therefore allowing the entire z-stack to be analyzed. The images were fed into our custom analysis pipeline and using the nuclear, MitoHealth, and MitoTracker channels, the number of cells was determined for each. Viable and non-viable cells were determined using colocalization of the nuclear channel and the viability dye images after thresholding. MitoHealth and MitoTracker cell object intensity distributions were quantified using cellular objects positive for MitoHealth and MitoTracker. Statistical analysis was performed using GraphPad Prism statistical software.

### Analysis

The HCT-116 spheroids were treated in 5 conditions to determine whether the C12-Ether-PEG-1000 and C18-Amide-PEG-1000 conjugates were found to have an effect.

The results show a statistical analysis of cell viability, mitochondrial membrane potential, and signal intensity of MitoHealth and MitoTracker stains. Analysis of viable cell counts shows a decrease in number of viable cells as compound concentration increases in the C-12 and C-18 treatments. Calculated IC50 values were 60.50 and 32.73 μM, respectively. Notably, non-conjugated Lauric Acid and Polyethylene Glycol showed no difference between treatment and control on cell viability. All other conditions show no significant difference between treatment and control on cell viability. The counting of viable cells through Viability, MitoHealth, and MitoTracker staining reveal similar results. MitoTracker intensity increased drastically at highest concentrations of C-12 and C-18. Raw Cell Counts and Percent Viability are represented as cells not positive for Viability stain. Raw Cell Counts for MitoHealth and MitoTracker were evaluated by quantifying the number of cells positive for the respective stain.

A series of images was obtained after spheroids containing HCT-116 cells were incubated with 300 μM concentrations of C12-Ether-PEG-1000, C18-Amide-PEG-1000 and various controls. Treatment of HCT-116 cells in 2D model with the test articles showed strong effects on cell viability. An additional unanticipated discovery showed differences between the targeting agents selected with C12 fatty acid being substantially less effective than C18 fatty acid, despite the C-18 conjugation using the more labile ester chemistry, to PEG molecular weight 1000 as visualized by the Phalloidin stain and viability staining. The fatty acids and PEG only controls or mixtures of the non-conjugated PEG-1000 and fatty acid showed no activity confirming the requirement for the conjugate to be present and stable to achieve the desired results. Monolaurin (ML) was tested as a non-PEG lauric acid (C12) control.

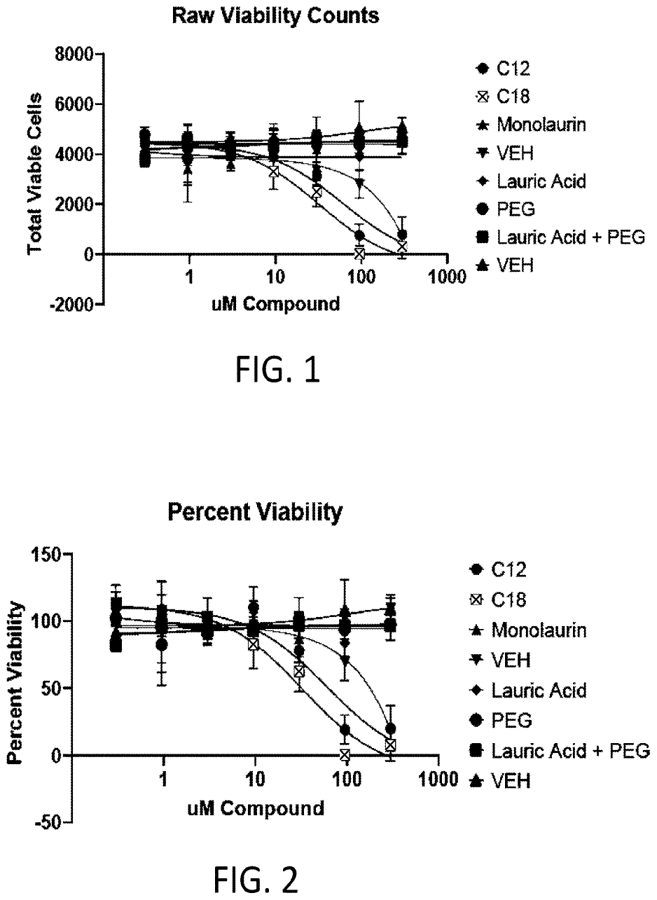

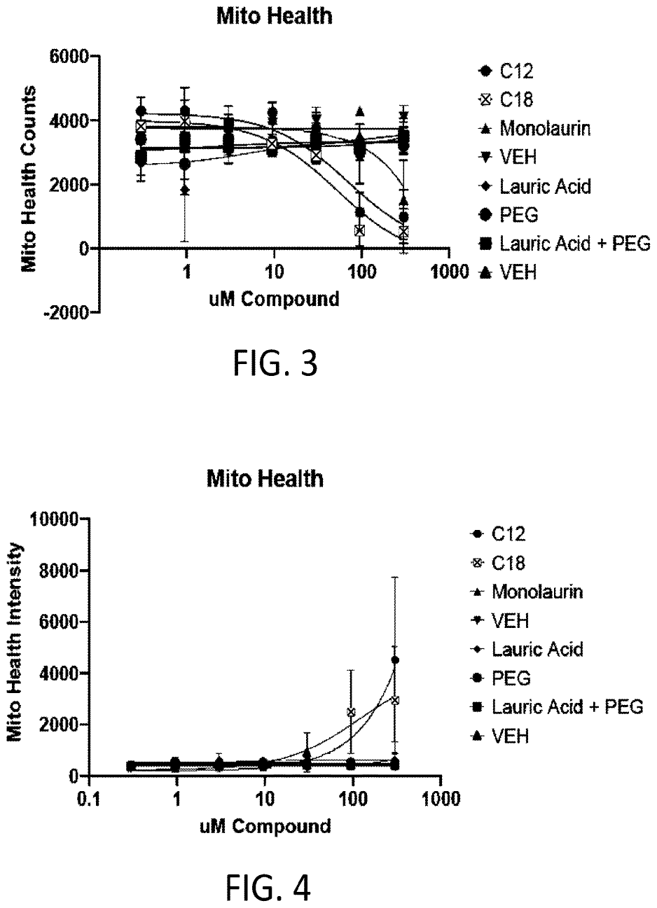

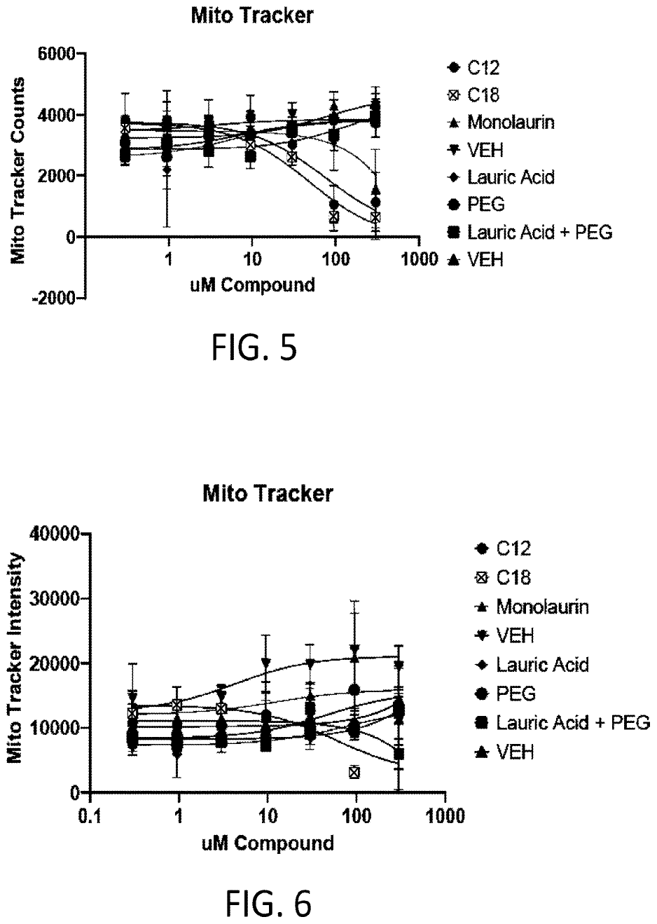

FIGS. 1-6 illustrate the statistical analytical significance of the data, demonstrating that C12-Ether-PEG-1000 and C18-Amide-PEG-1000 effectively kill cancer cells with C18-Amide-PEG-1000 preferred. In FIGS. 2 and 3, the treatment of HCT-116 spheroids with conjugated compounds C-12 and C-18 show a significant effect on cell viability. No other treatment showed significant effects on cell viability, non-conjugated Lauric Acid and Polyethylene Glycol included. This shows the conjugation of Lauric Acid with Polyethylene Glycol, rather than a mixture of Lauric Acid and Polyethylene Glycol, influences cell viability. Although not quantified, an effect on spheroid size in C-12 and C-18 treatments can be observed as well, another indicator of cell health and viability.

MitoHealth and MitoTracker data in FIGS. 3-5 show similar favorable data for the inventive conjugates. MitoHealth intensities remained consistent until the two highest concentrations where values increased.

MitoHealth accumulates in the mitochondria of live cells proportional to mitochondrial membrane potential. MitoTracker intensity shows a drop off at the highest concentrations of the C-12 and C-18 conjugates. MitoTracker passively diffuses across the plasma membrane and accumulates in the active mitochondria of a cell. There is a clear effect on Viability from the conjugated compounds.

### Example 2

In this example, 2 separate PEG-stearic acid conjugates (C18=C18-Ether-PEG-1000) were used to demonstrate cell kill activity using HCT-116 cells. The conjugates used in the study were polyethylene glycol octadecyl ether having the structures

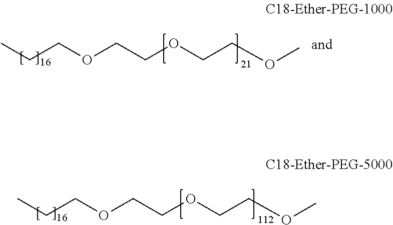

Stearic alcohol was conjugated to either mPEG-1000, designated C18-Ether-PEG-1000, or mPEG-5000, designated herein as C18-Ether-PEG-5000; with n being an integer equal to the number of repeating units of ethylene oxide (44 mass unit per ethylene glycol) sufficient to provide the desired molecular weight.

Cells were incubated for 72 hours in a 37° C./5% CO2 incubator. C18-Ether-PEG-1000 doses were administered via I.DOT dispensing platform while C18-Ether-PEG-5000 doses were administered through a multi-channel automatic pipette. At the conclusion of the 72-hour timepoint, cells were labeled with 6 μL/mL MitoTracker Deep Red and 1:250 Viability Dye.

Following labeling, cells were fixed in 10% neutral buffer formalin and permeabilized with 0.2% Triton X-100 in 1×PBS. Cells were then labeled with 1:500 Hoechst 33342 Ready Flow™ Reagent And 5 μL/mL Alexa Fluor 488 phalloidin.

Cells were then imaged at 20× widefield using a Molecular Devices ImageXpress Micro Confocal High-Content imaging platform. The scan laser/dichromic settings were tuned for 405, 488, 647, and 730 nm excitation wavelengths. Images were acquired and saved as 16-bit 2048×2048 TIF files for further processing. A grid of the sites at well centers was acquired during image capture.

The images were then analyzed using nuclear, phalloidin, and MitoTracker Channels. The number of cells was determined for each. Viable and non-viable cells were determined using colocalization of the nuclear channel and the viability dye images after thresholding. Phalloidin and MitoTracker cell object intensity distributions were quantified using cellular objects positive for phalloidin and MitoTracker. Statistical analysis was performed using GraphPad Prism statistical software. Nonlinear regression was used as a curve fitting tool to determine compound efficacy.

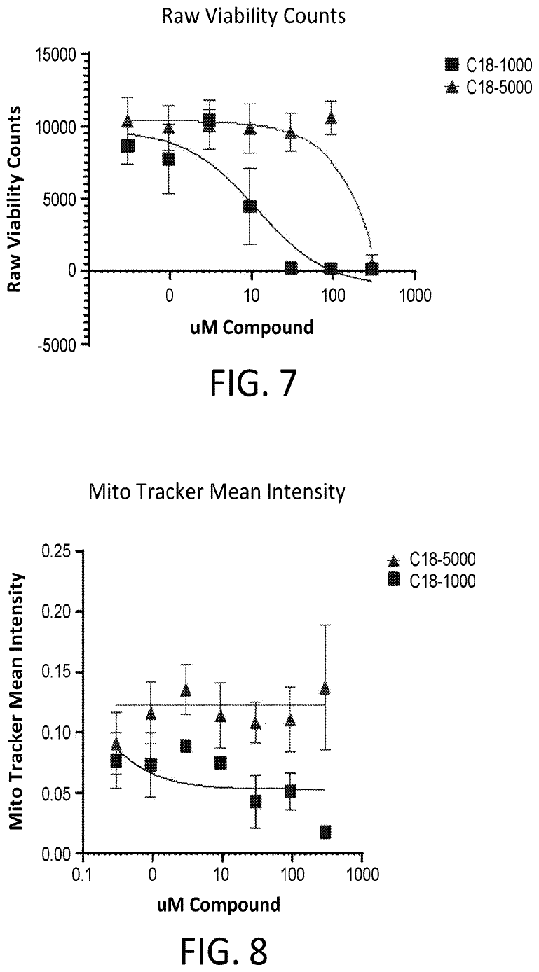

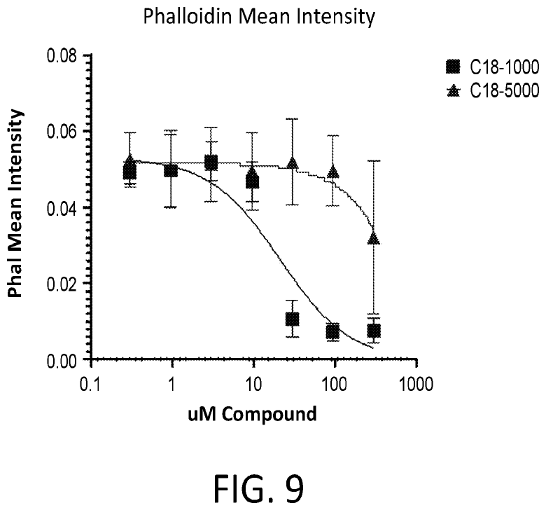

FIGS. 7-9 show treatment of HCT-116 cells in 2-D model with the C18-Ether-PEG-1000 showed strong effects on cell viability, but greater effect than C18-Ether-PEG-5000. A surprising result observed was that C18-Ether-PEG-5000 reached a trigger concentration leading to death of all the cells present at 100 μM in contrast to C18-Ether-PEG-1000 which showed an increasing level of cell death as drug concentration increased where total cell death occurred at 50 μM. This suggests some differences in mechanisms of action of the two compounds based on the PEG molecular weight.

MitoTracker intensities showed no effects in relation to the C18-Ether-PEG-5000 compound while the C18-Ether-PEG-1000 had a modest effect, suggesting the primary intracellular site of activity to be the cytoplasm. However, Phalloidin intensities did show a relationship to shifts in compound concentrations. As compound concentration increased, phalloidin intensities decreased. Suggesting involvement of the cytoskeleton in the compound mechanism of action, suggesting an additional difference between the two compounds emphasizing the importance in consideration of a conjugated structure of polymer, molecular weight, geometry, and chemical linkage.

### Example 3

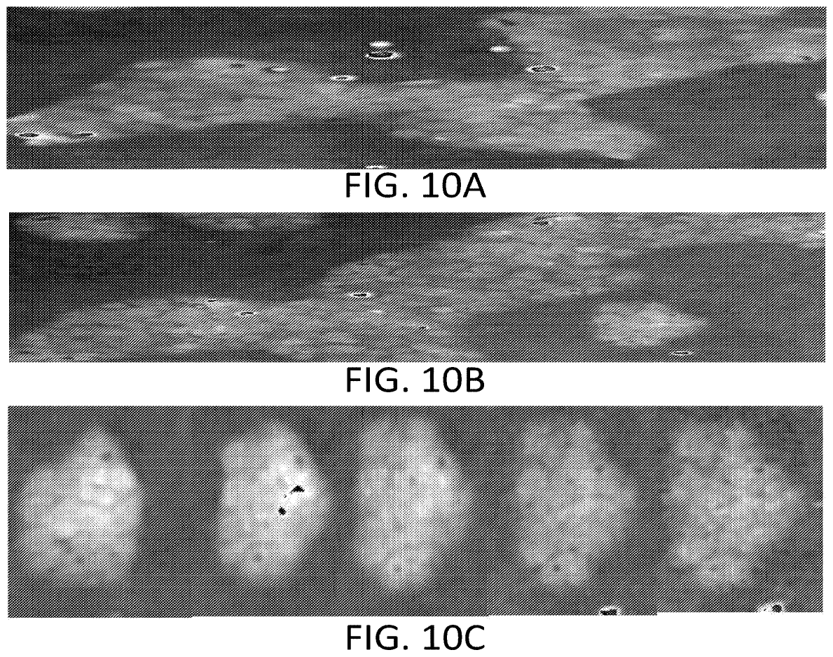

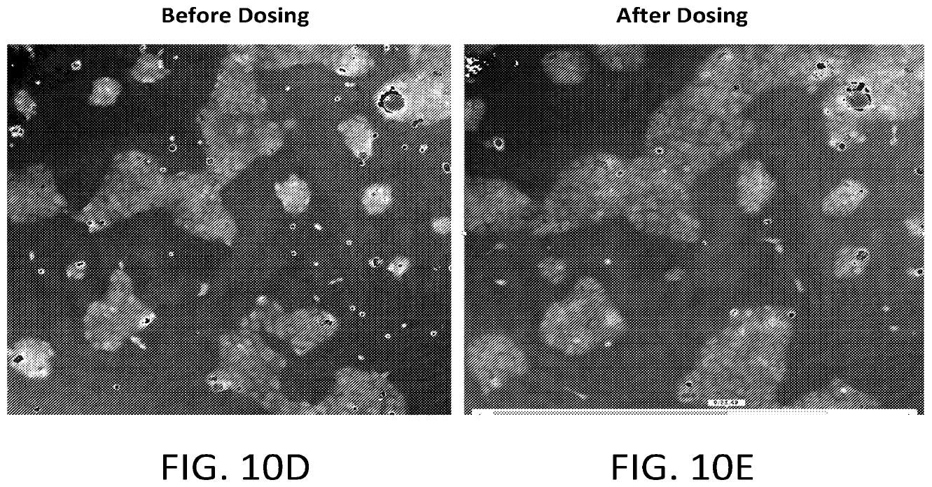

In this example, human pancreatic cancer (BXPC3) cells were grown on microscope slides for 24 hours prior to administration of test articles. Both individual cells and clusters of cells forming tumors can be seen microscopically. Imaging was at 10× with photographs taken once every five minutes. Turning now to FIG. 10A, cell clusters are shown prior to addition of drug, illustrating a characteristic mass of tumor cells (BxPC-3) prior to dosing, with cells are tightly clustered, and individual cells are migrating towards, and amalgamating with the forming mass. In FIG. 10B, the same clusters are shown after dosing and incubating after 19 hours, illustrating the effects of drug on an existing mass and migration and further accumulation of cells. Cells show a visible increase in volume due to an influx of water (as determined by dry mass figure, see Example 9, infra. Not to be bound by theory, water, and ion flux from outside the cells is due to the oncotic pressure gradient produced by cell uptake of the drug. As shown in FIG. 10B, cellular swelling contributes to a dissociation of the accumulated mass and loss of cellular motility and death. In FIG. 10C, a smaller cluster in 5-hour intervals is shown, illustrating a time sequence of the events shown in FIG. 10B for a single cluster in field of view. Time between imaging is approximately 43 hours. Dosing was with C18-Ether-PEG-5000 at 300 μM. FIGS. 10D and 10E provide additional views of the cells before dosing and after dosing, illustrating the swelling of individual cancer cell and clusters, pre and post C18-Ether-PEG-5000 dosing for the entire field of view.

### Example 4

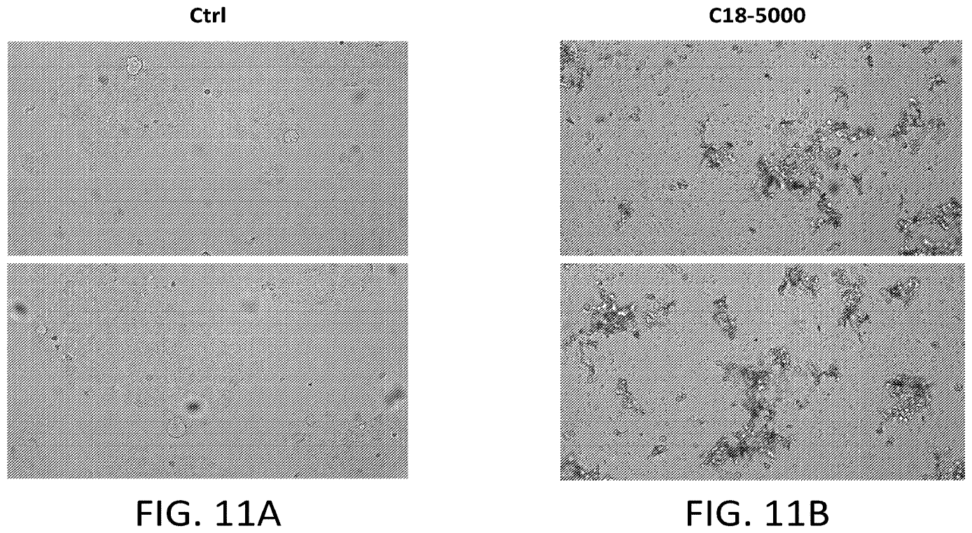

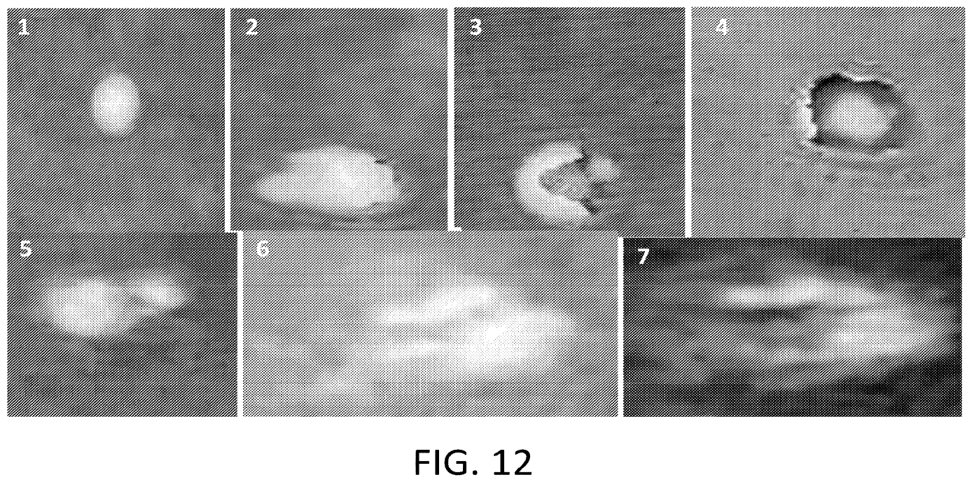

In this example, treatment of human pancreatic cancer (BXPC3) cells with C18-Ether-PEG-5000 was compared to a no drug control. Turning now to FIGS. 11A and 11B which are microscopic photographs, it can be seen that after 24 hours of incubation, the cells treated with the C18-Ether-PEG-5000 were completely destroyed whereas the control provided no evidence for activity. FIG. 11A illustrates cells prior to dosing and FIG. 11B shows post dosing and only cellular debris remaining after 24 hours of incubation with 300 μM drug.

### Example 5

In this example, human pancreatic cancer (BXPC3) cells were incubated with 200 uM of C18-Ether-PEG-1000. FIG. 12 is a series of photographs taken over a period of 1 hour and illustrates cell fragmentation within 60 minutes of dosing for C18-Ether-PEG-1000. Each photograph shows a single human pancreatic cancer (BXPC3) cell from initiation after addition of C18 1000 to a concentration of 200 micromolar. The timeline proceeds from left to right. The progression of photos shows the single cell initially contorting, showing protein precipitation and eventually cell death. Surprisingly, a kill kinetic much more rapid than the C18-Ether-PEG-5000 analogue (19 hours) is found along with differences in the degree of cellular swelling observed. In picture 2 of FIG. 12, cells are shown to be contorting in response to drug prior to death. Pictures 3 and 4 of FIG. 12 further illustrate changes in protein solubility and precipitation (molecular crowding) as well as membrane disruption. This reaction is most likely due to changes in oncotic/osmotic pressure, dielectric constant, and molecular crowding.

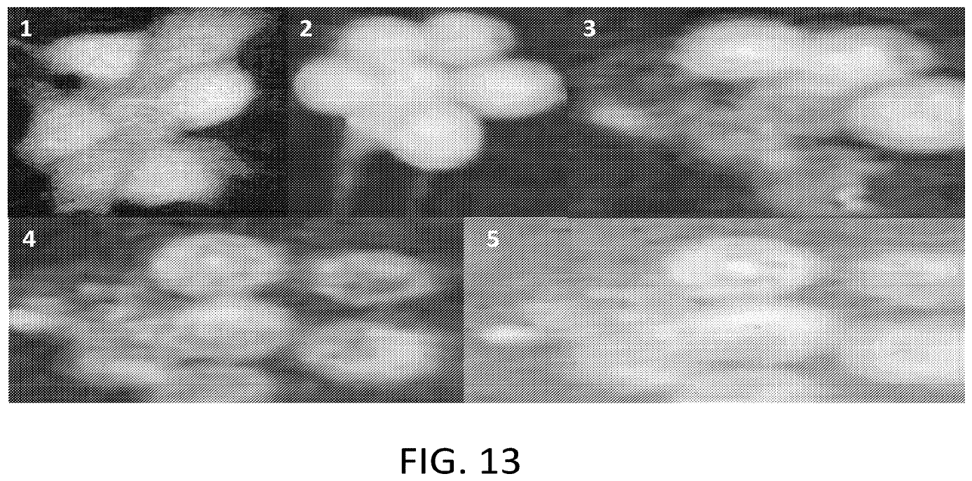

FIG. 13 provides a related observation of a cluster of human pancreatic cancer (BXPC3) cells with images taken over a 1-hour period, with a timeline proceeding from left to right shows membrane disruption and cell death. After addition of C18-Ether-PEG-1000 at a concentration of 200 μM. Events parallel those described for a single cell C18.

### Example 6

In this example, a comparison of 100 μM vs 200 μM C18-Ether-PEG-1000 against human pancreatic cells (BXPC3) cells after 24 hours of incubation was demonstrated. The effect was drastic with both concentrations, however there was less cell kill at the lower concentration.

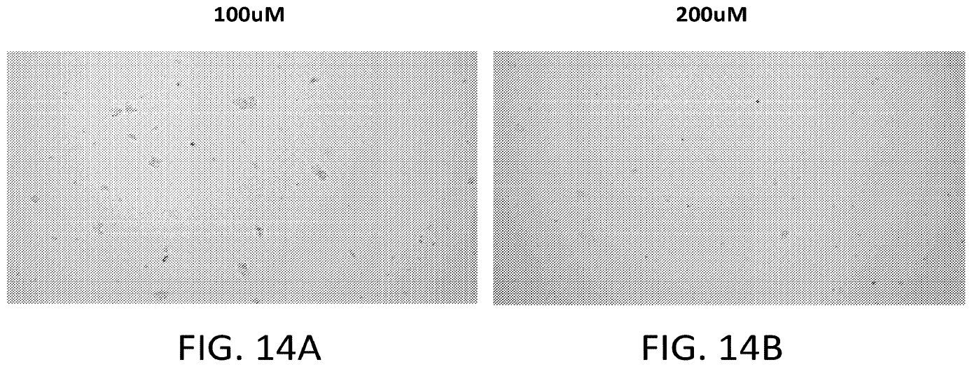

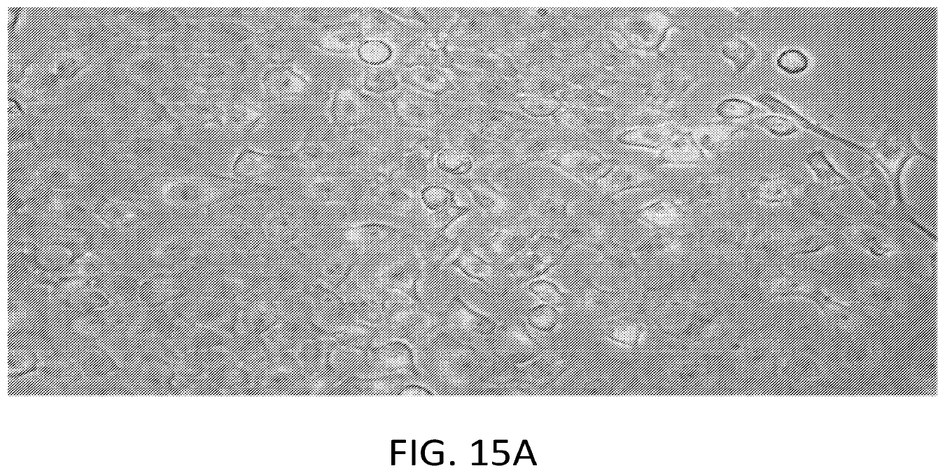

FIGS. 14A and 14B illustrate dose dependent effects for the C18-Ether-PEG-1000 analogue. Increasing drug concentration is proportional to cell death over the same period of incubation. C18-Ether-PEG-1000 cluster cells at a higher magnification demonstrated membrane disruption and molecular crowding effects seen by changes of phase contrast.

### Example 7

In this example, human pancreatic cancer (BXPC3) cells were incubated with 40 micromolar. MeOPEG-1000-NH₂ conjugated to carboxytetramethylrhodamine (TAMRA) mixture structure shown below. TAMRA is a known structure that accumulates in mitochondria.

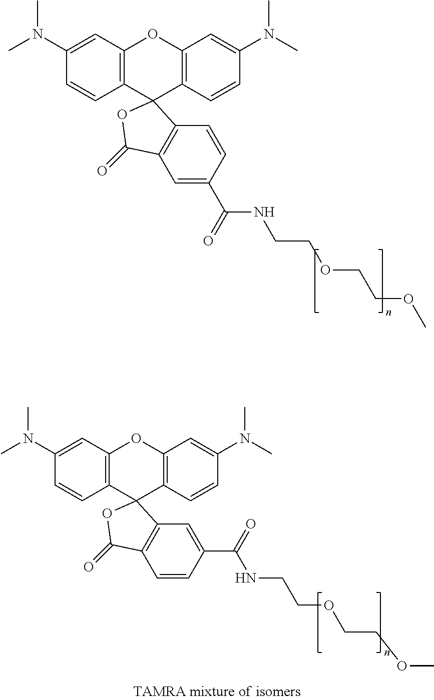

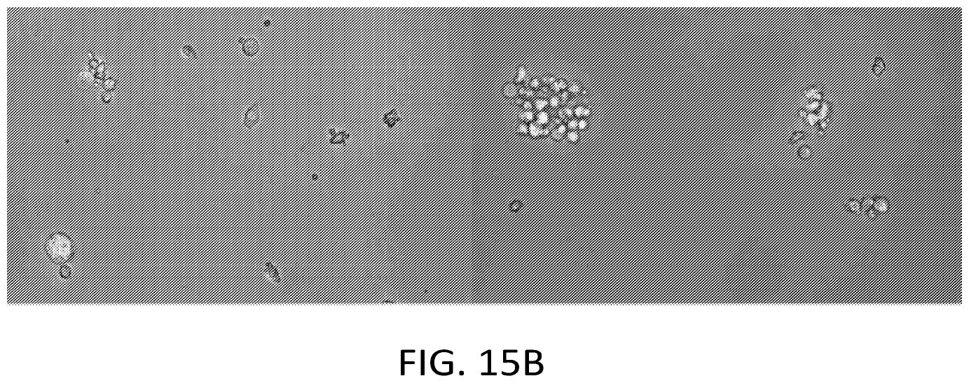

Cells were imaged at 40× microscopically after 24 hours. See FIGS. 15A-15B. FIG. 15A demonstrates that at low concentration (40 μM), the cells appear to be viable, and the fluorescence is located around the nucleus, fluorescence presumably in mitochondria. In FIG. 15A drug cell death was apparent, and florescence distributed throughout the cells in and around the nuclei suggesting leakiness or rupture of mitochondria. However, at 200 μM, nearly all cells were killed or dying. Substituted rhodamine differs from PEG fatty acid analogues in that cellular uptake is dependent on mitochondrial-plasma membrane potential differences such that accumulation in mitochondria occurs because of molecular charge.

FIG. 15A illustrates rapid uptake into cells with drug presumably localized predominately to mitochondria as fluorescence is not seen in the nucleus. At this concentration however some level of cell death is observed with greater levels seen as the drug concentration increases suggesting a dose response relationship. Surprisingly, no observation of inhibited motility or cell paralysis was made during drug incubation even at higher concentrations. In FIG. 15B, nearly all cells are dead after 24 hours (200 μM) with remaining dying cells showing some residual fluorescence throughout the remaining cells. Demonstrating that choice of targeting agent influences site of intracellular delivery and concentration and subsequent effects of the polymer.

### Example 8

In this example, human pancreatic cancer (BX PC3) cells were evaluated for motility under conditions of control (no drug) or addition of C18-Ether-PEG-1000 (200 micromolar) for 24 hours or C18-Ether-PEG-5000 (300 micromolar) for 6 hours.

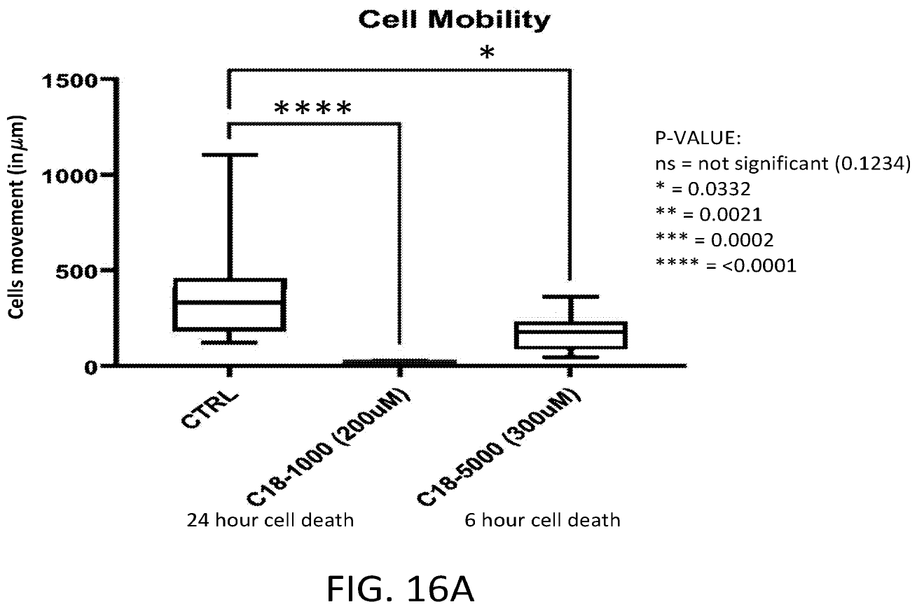

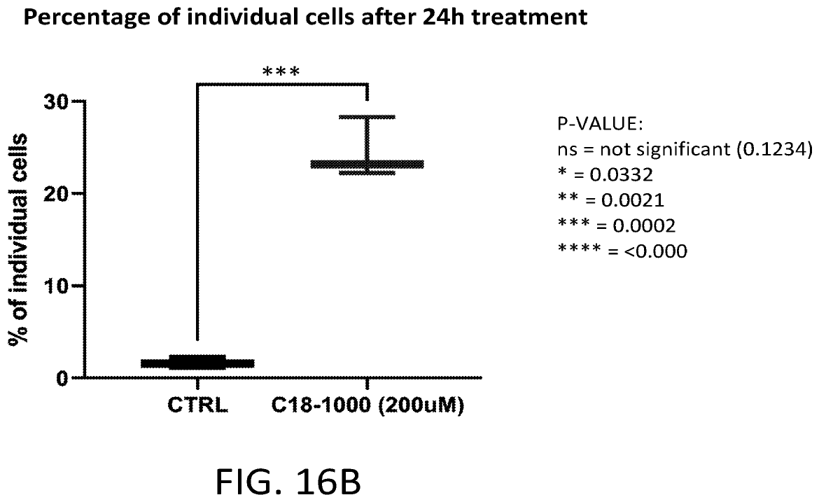

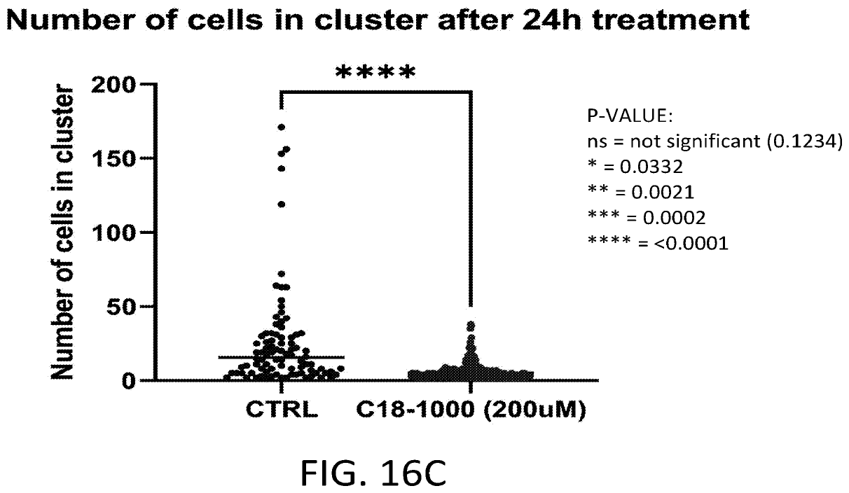

FIG. 16A is a graph that illustrates the effects on cell motility of C18-Ether-PEG-1000 and C18-Ether-PEG-5000. C18-Ether-PEG-1000 at 200 μM after 30 minutes inhibited motility with P-values compared to control of 0.0001 and for C18-Ether-PEG-5000 at 300 μM after 6 hours had a P-value of 0.0332. The differential effects of the inhibition of cellular motility and kinetics lead to a reduction of metastatic disease. The difference in kinetics of effects for the two drugs is surprising due to the differences in the molecular weight. The surprising temporal difference between cell paralysis and death and differences between 1000 and 5000 MW suggest multiple effects that would be beneficial to inhibiting spread of disease in addition to cell killing. Cell motility is a fundamental requirement for tumor growth and metastasis as cells invade proximal and distal tissues.

FIG. 16B illustrates examination of drug ability to effect cell cluster formation, an important aspect of metastatic disease. During incubation, BX PC3 cells associate into clusters for formation of tumors. The numbers of cells participating in clusters visible in the microscopic field are shown for a no drug added control and for C18-Ether-PEG-1000 (200 micromolar) treatment after 24 hours. It can be seen that after treatment, the number of cells in clusters is significantly reduced. The reduction of the cluster development in C18-Ether-PEG-1000 substantiates the FIG. 16B analysis, that the reduction of cell motility reduces cluster formation and metastatic cancer. Differences most likely reflect the level of oncotic pressure change associated with polymer molecular weight and degrees of uptake as well as sites of accumulation and the specifics of molecular crowding effects.

FIG. 16C provides further evidence of the effectiveness of the treatment and lack of tumor cell cluster formation. Note the significant number of cells remaining as individual cells rather than participating in cluster formation when comparing the control and the C18-Ether-PEG-1000 (200 micromolar) treated cells after 24 hours.

### Example 9

In this example, the change in dry mass detected by holographic microscopy post dosing of C18-Ether-PEG-1000 ether of BXPC3 cells was determined. Cancer cells remain stable prior to dosing and post dosing, an influx of dry mass is observed. Post dosing within 40 minutes, membrane disruption occurs leading to cell death.

FIG. 17A illustrates how the dosing of C18-Ether-PEG-1000 drug changes in intracellular dry mass on addition of drug and subsequent release of intracellular dry mass contents after rupture of cell membranes. The global phase fluctuation graph for C18-Ether-PEG-1000 showed, while before dosing the phase signal remain stable and a phase drop in dry mass approximately 30-50 minutes post dosing. Cell mass dropped by 3-fold, showing molecular crowding influencing membrane disruption and rupture and a substantial release of intracellular dry mass contents. FIG. 17B illustrates by zooming in on the region before the major drop, that 1) the signal drops slowly at the beginning; 2) the signal is steady for about 30 minutes and 3) the signal gets higher almost immediately after addition of C18-PEG-1000 Ether and drops slowly before the major drop.

FIG. 17C illustrates stabilization of dry mass in control cells prior to C18-Ether-PEG-1000 addition, showing no molecular crowding event prior to C18-Ether-PEG-1000 dosing.

FIG. 17D illustrates a repeat study of C18-Ether-PEG-1000 showing changes in intracellular dry mass on addition of drug and subsequent release of intracellular dry mass contents after rupture of cell membranes. There was a major phase drop about 25 minutes after the addition of the drug. Cell mass decreased by a factor of 2. There was perturbation of the flow starting between 34-38 minutes and an increase of signal after the drug effect. By zooming in on the region before the major drop, FIG. 17E illustrates that 1) the signal is stable before (33 minutes) 2) there is a signal drop due to flow change perturbation and 3) the signals get higher after the addition of the drug with no drop before rupture. FIG. 17F is similar to FIG. 17C, illustrating stabilization of dry mass in control cells prior to drug addition.

### Example 10

In this example, the anti-tumor effects of C18-Ether-PEG-5000 and C18-Ether-PEG-1000 on the proliferation and cytotoxicity of solid and hematologic cell lines is demonstrated using 6 concentrations on A549, HL-60, Raji B, Panc-1, and PC3 cancer cell lines.

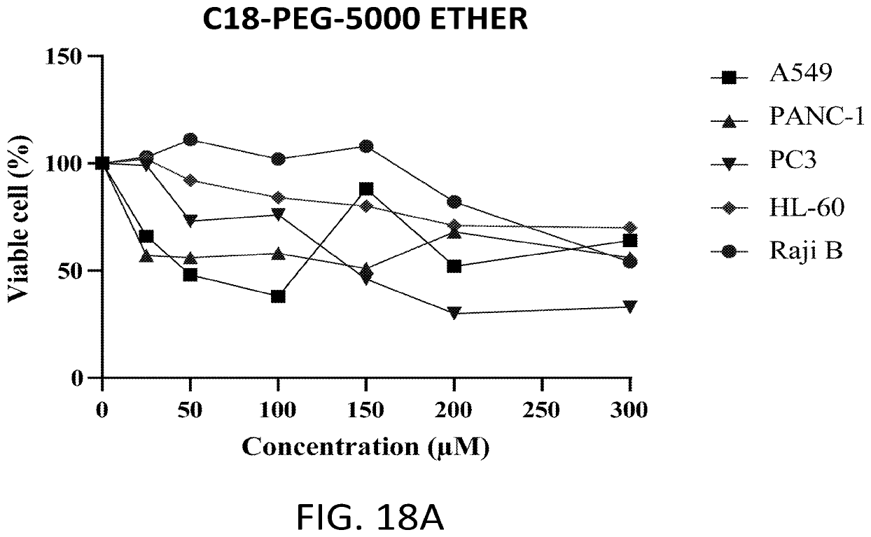

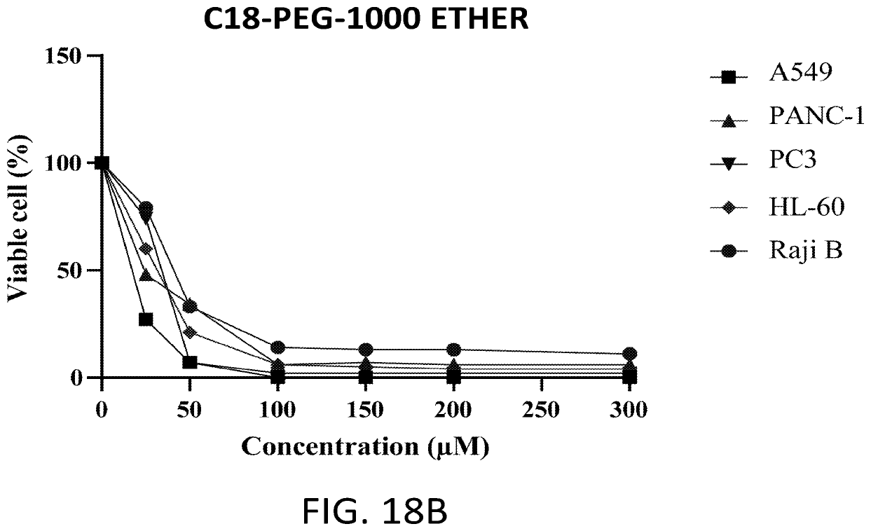

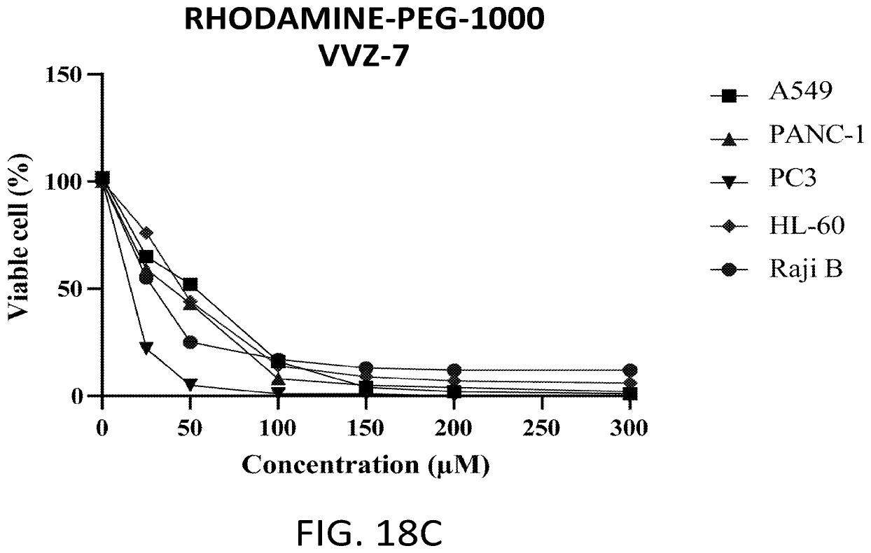

FIG. 18A illustrates anti-cancer activity against cell lines shown for C18-Ether-PEG-5000. In 5 different cell lines of both hematologic and solid cancer cell lines, a reduction in cell viability of up to 50 percent in a 4-5 h period at 300 μM was observed.

FIG. 18B illustrates anti-cancer activity against cell lines shown for C18-Ether-PEG-1000. In 5 different cell lines of both hematologic and solid cancer cell lines, a reduction in cell viability at 80% cell death at 50 μM and at 100% cell death at 100 μM in a 4-5 h period was observed. This unanticipated result shows how C18-Ether-PEG-1000 demonstrates a more effective kill and a lower concentration for all solid and hematologic cell lines.

FIG. 18C illustrates anti-cancer activity against cell lines shown for PEG-1000-Amide-Rhodamine in 5 different cell lines of both hematologic and solid cancer cell lines. While not surprising given the proposed mechanism of action that any cell line possessing the receptors and transporters targeted such moieties in the embodiments of the invention, it was surprising that the differences in PEG molecular weight and the fatty acid targeting agent does result in substantial differences in ability to promote cancer cell death. This suggests that heterogeneity of response may be present across different cell types add the benefit of use of mixtures of PEG conjugates and drugs in combination. Combinations of drugs may be necessary to kill all or the majority of the cancer- and cancer-causing cell types in a patient.

### Example 11

In this example, a Cypre PDX assay was carried out in 5 different pancreatic cancer patients using 5 test compounds. C18-ETHER-PEG-1000, C18-PEG-5000, C18-Squaramide-PEG-1000 (C18S-1000), 9D-Z-C18-Squaramide-PEG-1000 (C18SU-1000), PEG-1000-Amide-Rhodamine were tested in the Cypre 3D Oncology in vitro patient-derived xenograft (PDX) Panel using standard 5 PDX derived cell line models in co-culture with human dermal fibroblasts (HDF) in order to screen for therapeutic effect. The endpoint from this assay was tumor size and DRAQ7 staining.

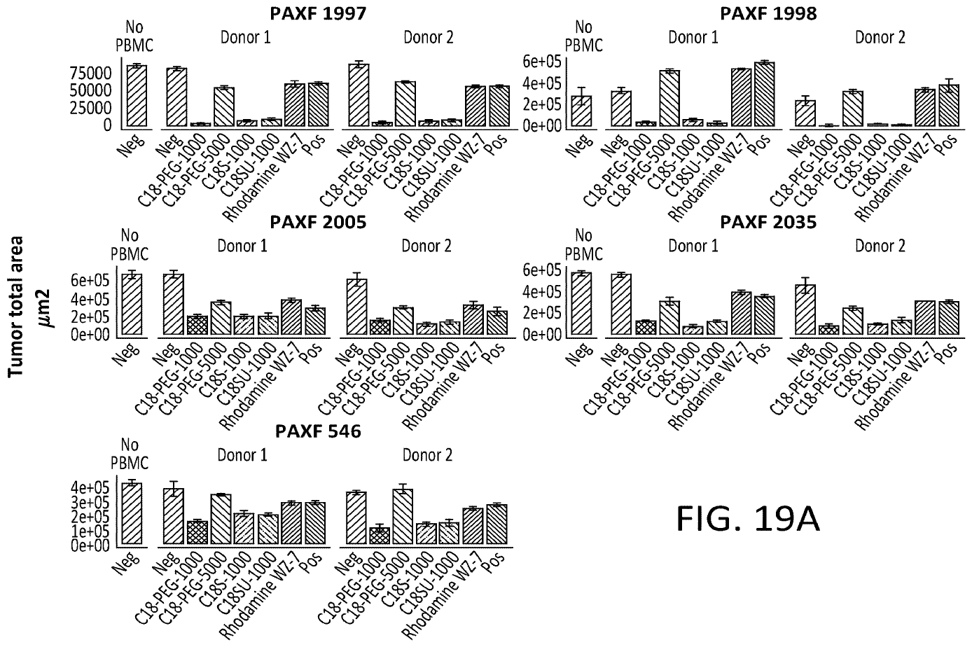

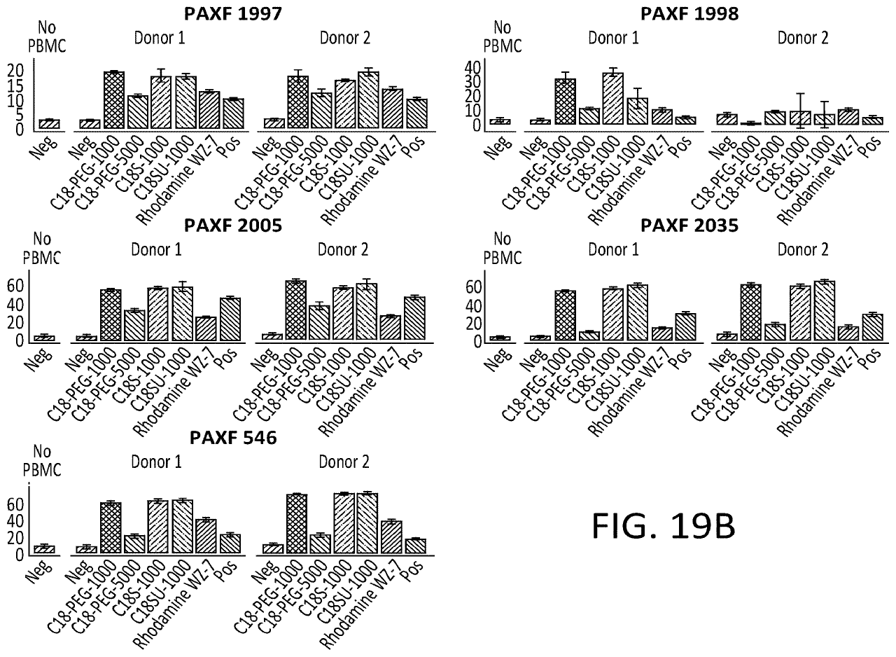

FIG. 19A illustrates the effect of test compounds on tumor total area evaluated in the PDX samples taken from 5 independent pancreatic patients compared to staurosporine (a non-selective kinase inhibitor) as a positive control.

PDX Tumor cells were expanded in 2D cell culture and then grown in 3D hydrogels, co-embedded with human dermal fibroblasts (HDF). Tumor-Fibroblast co-cultures were grown to sufficient size and treated with, C18-Ether-PEG-1000, C18-Ether-PEG-5000, C18-Squaramide-PEG-1000 and 9D-Z-C18-Squaramide-PEG-1000 for four days. After, 3D assays were stained with Hoechst and DRAQ7, fixed, and analyzed using a high content imager and proprietary image analysis software. Figure A shows a Tumor Total area assay where C18-Ether-PEG-1000 is compared to staurosporine, a positive control in a pancreatic assay in five human pancreatic patients. In each of the 5 patients, C18-Ether-PEG-1000 significantly outperformed standard of care (POS) with a further reduction of tumor inhibition of growth and area and cell kill by 2 to 6-fold. An unanticipated result shows C18-Ether-PEG-5000 to be equal to standard of care but not to C18-Ether-PEG-1000 nor to other analogs. Another unanticipated and significant result is the similarity in activity for the Squaramide-C18-PEG-1000 and Squaramide-9D-Z-C18-PEG-1000. Reports in the literature suggest that unsaturated analogs of fatty acids have little to no activity against solid tumors.

FIG. 19B illustrates the effect of test articles in comparison to a positive control (staurosporine) with cell death measured by DRAQ7 assay. Figure B shows a DRAQ7 cell death assay where, C18-Ether-PEG-1000 is compared to Staurosporine, a positive control in a pancreatic assay in five human pancreatic patients. In each of the 5 patients, C18-Ether-PEG-1000, Squaramide-C18-PEG-1000, and Squaramide-9D-Z-C18-PEG-1000 significantly outperformed standard of care (POS) with a reduction of tumor inhibition and area by 10 to 20-fold. An unanticipated result shows C18-Ether-PEG-5000 to be equal to standard of care.

### Example 12

In this Example, a PK analysis of the ether-linked stearic acid-PEG1000 (C18-Ether-PEG-1000) formulated both in 120 mg/kg in two routes of Iv and IP for three days was carried out. A second group of C-18 1000 ether was formulated in 200 mg/kg in IV for four days. The third group of C-18 1000 ether was formulated in 400 mg/kg and IV and 200 mg/kg in IP for four days was developed.

### PK Analysis of Invention Embodiments

Purpose: To collect samples after an injection of C18-Ether-PEG-1000 for PK analysis in female C57BL/6 mice.

### Drugs and Treatment:

| | | Formulation | Active | | | Dosing volume |
|---|---|---|---|---|---|---|
| Gr. | N | dose | dose | Route | Schedule | scale ml/kg |
| 1 | 3 | 120 mg/kg | 120 mg/kg | iv | qd × 3 | 10 |
| | 3 | 120 mg/kg | 120 mg/kg | ip | qd × 1 day 8 | 20 |
| 2 | 3 | 200 mg/kg | 200 mg/kg | iv | qd × 3 | 10 |
| 3 | 3 | 400 mg/kg | 400 mg/kg | iv | qd × 3 | 10 |
| | 3 | 200 mg/kg | 200 mg/kg | ip | qd × 1 | 20 |
| | | | | | day 8 | |

The 9 CR female C57BL/6 mice were randomized into treatment groups based on Day 1 bodyweight. Endpoint Day 5. All animals euthanized.

Summary of Mouse Plasma Sample Concentrations

| Group 2, | | | | | | |
|---|---|---|---|---|---|---|
| 200 mg/kg, IV | FP36 Concentrations (ng/mL) in Mouse Plasma | | | | | |
| Time Points | Animal ID | | | | | |
| (hrs) | 1 | 2 | 3 | Mean | SD | % CV |
| 0.167 | 463000 | 359000 | 86600 | 302867 | 194377 | 64.2% |
| 1.00 | 227000 | 116000 | 16100 | 119700 | 105499 | 88.1% |
| 3.00 | 159000 | 147000 | BQLa | 153000 | NA | NA |
| 8.00 | INS | INS | BQLa | NA | NA | NA |
| 24.0 | BQLa | BQLa | BQLa | NA | NA | NA |

BQLa = Below Quantitation Limit (<10,000 ng/mL)
BQLb = Below Quantitation Limit (<100,000 ng/mL)
NA = Not Applicable
INS = Insufficient sample for analysis

A preliminary evaluation of the pharmacokinetics of C18-Ether-PEG-1000 suggested an optimal tolerated dose 200 milligrams per kilo. During this time, the C_{Max} was very rapidly achieved and maintained even out to approximately 4 hours. This is an important consideration since studies have demonstrated that a pharmacodynamic requirement for optimal cancer cell kill is in the four-hour timetable. The concentrations achieved based on characterization of drug activity in cell culture studies indicate that the duration and level maintained is sufficient to provide substantial tumor and cancer cell kill.

### Example 13

### C18-Squarate-PEG-1000

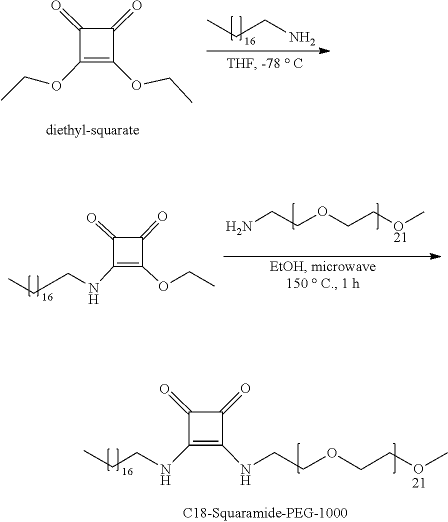

### 3-ethoxy-4-(octadecylamino)cyclobut-3-ene-1,2-dione

To a solution of 3,4-diethoxycyclobut-3-ene-1,2-dione (1.48 mL, 10 mmol, 1.0 equiv.) in THF (20 mL) at −78° C. under nitrogen atmosphere was added a solution of octadecan-1-amine (2.70 g, 10 mmol, 1.0 equiv.) in THF (20 mL) dropwise. After stirring for 20 minutes at −78° C., the reaction was allowed to warm to room temperature. After stirring for 1 hour at room temperature the reaction was determined to be complete by TLC (25% EtOAc/Hex). The reactions were filtered over celite wand washed through the celite with additional EtOAc. The filtrate was concentrated under reduced pressure and purified by silica gel chromatography (100% DCM to 25% EtOAc/DCM). Product containing fractions were concentrated under reduced pressure to give the title compound (3.88 g) as a white solid. (MH+) Not observed. ¹H NMR (500 MHz, CDCl3) δ 5.88 (s, 1H), 4.86-4.66 (m, 2H), 3.72-3.35 (m, 2H), 2.12-1.74 (m, 1H), 1.61 (p, J=6.7 Hz, 3H), 1.46 (t, J=7.2 Hz, 3H), 1.40-1.06 (m, 34H), 0.88 (t, J=7.0 Hz, 3H). 3-((2,5,8,11,14,17,20,23,26,29,32,35,38,41,44,47,50,53,56,59,62,65-docosaoxa-heptahexacontan-67-yl)amino)-4-(octadecylamino)cyclobut-3-ene-1,2-dione 3-ethoxy-4-(octadecylamino)cyclobut-3-ene-1,2-dione (95%, 2.0 g, 5.08 mmol, 1.0 equiv.) and 2,5,8,11,14,17,20,23,26,29,32,35,38,41,44,47,50,53,56,59,62,65-docosa-oxaheptahexacontan-67-amine (4.57 g, 4.57 mmol, 0.9 equiv.) were taken up in EtOH (50 mL) and heated for 1 hour in a microwave reactor for 1 hour at 150° C. The reaction was determined to be mostly complete by TLC (10% MeOH/DCM), staining with PMA. The crude reaction was concentrated under reduced pressure and purified by silica gel chromatography (0% to 5% to 10% MeOH/DCM). The column separated product with varying lengths of PEG chain. NMR's were taken of each batch. Batches that appeared to be clean product, with only variation at the length of PEG chain were combined and concentrated to give the title compound (1.9 g) as a white solid after several rounds of purification by silica gel chromatography.

¹H NMR (500 MHz, CDCl3) δ 6.70 (s, 1H), 6.58 (s, 1H), 3.63 (d, J=5.2 Hz, 99H), 3.37 (s, 3H), 1.56 (q, J=7.0 Hz, 2H), 1.25 (s, 33H), 0.87 (t, J=6.7 Hz, 3H).

The synthesis can be carried out using PEG-NH₂ of any desired molecular weight.

### Example 14

### 9D-Z-C18-Squaramide-PEG-1000

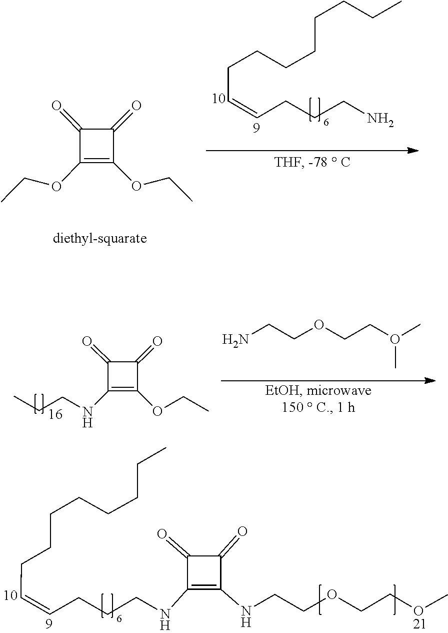

### (Z)-3-ethoxy-4-(nonadec-9-en-1-ylamino)cyclobut-3-ene-1,2-dione

To a solution of 3,4-diethoxycyclobut-3-ene-1,2-dione (0.870 mL, 5.88 mmol, 1.0 equiv.) in THF (12 mL) at −78° C. under nitrogen atmosphere was added a solution of (Z)-nonadec-9-en-1-amine (1.57 g, 5.88 mmol, 1.0 equiv.) in THF (5 mL) dropwise. After stirring for 1 hour at −78° C. the reaction was allowed to warm to room temperature and was determined complete by TLC (25% EtOAc/Hex). The crude reaction was concentrated under reduced pressure and purified by silica gel chromatography (10% to 25% EtOAc/Hex). Fractions containing clean product were combined and concentrated to give the title compound (1.55 g) as a sticky white solid. (MH+) 392.30. 1H NMR (500 MHz, CDCl3) δ 5.92 (s, 1H), 5.42-5.28 (m, 2H), 4.84-4.68 (m, 2H), 3.78-3.32 (m, 2H), 2.09-1.92 (m, 4H), 1.63-1.56 (m, 3H), 1.46 (t, J=7.0 Hz, 3H), 1.39-1.22 (m, 25H), 0.88 (t, J=6.5 Hz, 3H).

### (Z)-3-((2,5,8,11,14,17,20,23,26,29,32,35,38,41,44,47,50,53,56,59,62,65-docosaoxaheptahexacontan-67-yl)amino)-4-(nonadec-9-en-1-ylamino)cyclobut-3-ene-1,2-dione

To a solution of (Z)-3-ethoxy-4-(nonadec-9-en-1-ylamino)cyclobut-3-ene-1,2-dione (500 mg, 1.28 mmol, 1.0 equiv.) in EtOH (10 mL) and THF (5 mL) was added 2,5,8,11,14,17,20,23,26,29,32,35,38,41,44,47,50,53,56,59,62,65-docosaoxahepta-hexacontan-67-amine (95%, 639 mg, 0.64 mmol, 0.50 equiv.) followed by TEA (0.35 mL, 2.55 mmol, 2.0 equiv.). The reaction was stirred for 16 hours at 60° C. and cooled to room temperature. Purification by silica gel chromatography (0% to 5% MeOH/DCM) yielded the title compound (433 mg) as a white solid after concentration of product containing fractions.

1H NMR (500 MHz, CDCl3) δ 6.77 (s, 1H), 6.52 (s, 1H), 5.41-5.30 (m, 2H), 3.89-3.44 (m, 108H), 3.39-3.35 (m, 3H), 2.05-1.91 (m, 4H), 1.62-1.51 (m, 2H), 1.38-1.21 (m, 25H), 0.92-0.83 (m, 3H).

The synthesis can be carried out using PEG-NH₂ of any desired molecular weight.

### CONCLUSIONS

Compositions comprised of one or more conjugates of a cancer cell selective targeting uptake promotor, such as but not limited to an essential cancer nutrient linked to a modulator of molecular crowding and colloid oncotic pressure, such as a polymer are useful in the treatment of cancers and malignancies in mammals and against mammalian cancer cells. In most preferred embodiments, the linkages are chemically stable, resistant to extracellular and intracellular metabolism. Increased colloid oncotic pressure and molecular crowding is effectively delivered to the intracellular environment and compartments of cancer- and cancer-causing cells or exosomes by the cancer selective receptors and transporters.

The mixture of targeted moieties, colloid oncotic pressure and molecular crowding agents are selected on the basis of activity demonstrated against patient derived tumor tissues and cells. Additionally, the conjugates may be selected based on the cellular distribution and affinity of the mixture of the receptors and transporters for the targeting moieties. Maximizing the distribution among diseased and disease-causing cells of the pharmaceutical colloid oncotic pressure/molecular crowding agents by co dosing or independent dosing protocols.

All references cited herein are incorporated herein by reference in their entirety. To the extent publications and patents or patent applications incorporated by reference contradict the disclosure contained in the specification, the specification is intended to supersede and/or take precedence over any such contradictory material.

Where a range of values is provided, it is understood that the upper and lower limit, and each intervening value between the upper and lower limit of the range is encompassed within the embodiments.

1. A method of causing mammalian cell death, comprising contacting a mammalian cell with a conjugate of the Formula (I) T-L1-R
wherein T is a non-cleavable cellular uptake promoter or a targeting moiety,
L1 is an amide, ether or squaramide linker, and
R is a modulator of colloid oncotic pressure and molecular crowding;
under conditions sufficient to allow the conjugate to be taken up by the cell, causing cell membrane disruption and death of the mammalian cell.

2. The method of claim 1, wherein the targeting moiety is a cellular uptake promotor selected from the group consisting of fatty acids, fatty acid analogs, sugars, amino acids, peptides, protein, and lipids.

3. The method of claim 2, wherein the fatty acid is of the formula CH₃(CH₂)ₙCOO— where n is an integer of from about 2 to about 20.

4. The method of claim 1, wherein the conjugate of Formula (I) is selected from the group consisting of Formulae (II)-(VI): 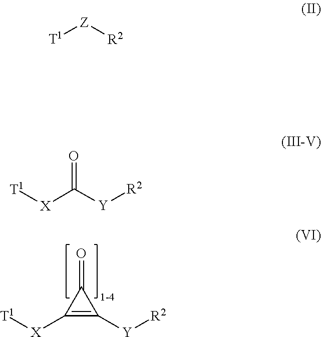
wherein:
T¹=C₈-C₂₂ alkane, C₈-C₂₂ unsaturated alkane (1-5 unsaturations, each unsaturation independently Z or E steroisomer);
X and Y are independently CR¹R³, O, NR¹, or S;
R¹ is H, C₁-C₆ alkyl, C₃-C₁₂ cycloalkyl, C₃-C₁₂ heterocycloalkyl with 1-3 heteroatoms, or OH;
R³ is H, C₁-C₆ alkyl, C₃-C₁₂ cycloalkyl, or C₃-C₁₂ heterocycloalkyl with 1-3 heteroatoms; wherein R¹ and R³ may be attached together to form a 3-, 4-, 5-, 6-, 7- or 8-membered ring with 1-3 heteroatoms;
Z is O, S, SO², SONH, Se, NR¹, (NR¹R³)⁺;
R²= 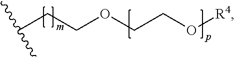
wherein m is an integer from 1 to 8, p is an integer from 15 to 120 and R⁴ is H, 1-7 atom alkyl, branched alkyl, cycloalkyl or heterocycloalkyl,
or R²=branched PEG chain MW<1500 or other polymer; and
R⁴ is H, 1-7 atom alkyl, branched alkyl, cycloalkyl or heterocycloalkyl.

5. The method of claim 1, wherein the conjugate of Formula (I) is selected from the group consisting of: 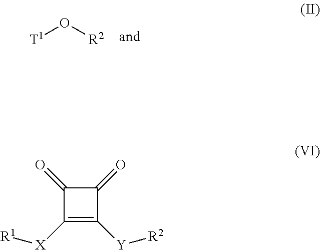
wherein T¹ is C₁₀-C₂₀ alkane, or C₁₀-C₂₀ unsaturated alkane (1-2 unsaturation, each independently Z or E);
X and Y are independently O, NR¹;
R¹ is H, C₁-C₆ alkyl, C₃-C₁₂ cycloalkyl, C₃-C₁₂ heterocycloalkyl with 1-3 heteroatoms, or OH;
R³ is H, C₁-C₆ alkyl, C₃-C₁₂ cycloalkyl, or C₃-C₁₂ heterocycloalkyl with 1-3 heteroatoms;
R²= 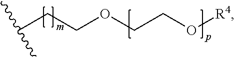
wherein m is an integer from 1 to 8; p is an integer from 20 to 25 and R⁴ is H, 1-7 atom alkyl, branched alkyl, cycloalkyl or heterocycloalkyl, or R² is branched PEG chain MW<1500 or other polymer.

6. The method of claim 1, wherein the conjugate of Formula (I) is selected from the group consisting of: 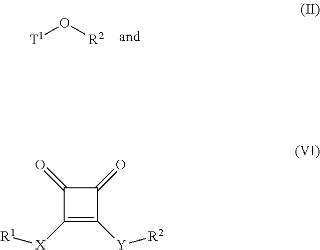
wherein T¹=C₁₀-C₂₀ alkane, C₁₀-C₂₀ unsaturated alkane (1-2 unsaturation, each independently Z or E);
X and Y are independently O or NR¹
wherein R¹ is H, C₁-C₆ alkyl, C₃-C₁₂ cycloalkyl, C₃-C₁₂ heterocycloalkyl with 1-3 heteroatoms, or OH;
R²= 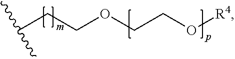
wherein m is an integer from 1 to 8, p is an integer from 19 to 22 and R⁴ is H, 1-7 atom alkyl, branched alkyl, cycloalkyl or heterocycloalkyl, or R² is branched PEG chain MW<1500 or other polymer.

7. The method of claim 1, wherein the conjugate corresponding to Formula (I) is selected from the group consisting of 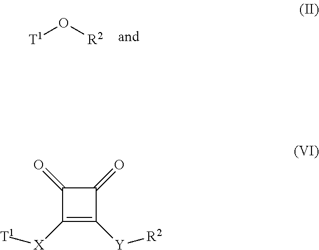
wherein
R²= 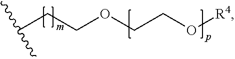
wherein m is an integer from 1 to 8, n is an integer from 19 to 22, or R²=branched PEG chain MW<1500 or other polymer;
T¹ is C10-C20 alkane, C10-C20 unsaturated alkane (1-2 unsaturation, each independently Z or E);
X and Y are independently O or NR¹;
wherein R¹ is H, C₁-C₆ alkyl, cycloalkyl, heterocycloalkyl with 1-3 heteroatoms, or OH; and
R⁴ is H, 1-7 atom alkyl, branched alkyl, cycloalkyl or heterocycloalkyl.

8. The method of claim 1, wherein the conjugate is selected from the group consisting of 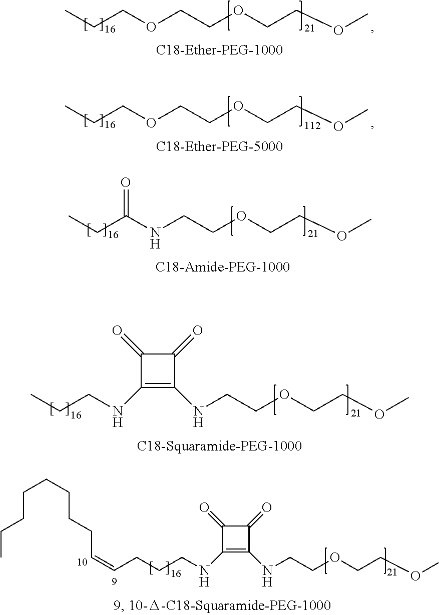
CH₃(CH₂)ₙCOO-L1-R, CH₃(CH₂)ₙCH₂O-L1-R,
CH₃(CH₂)ₙCONH-L1-R, and CH₃(CH₂)ₙCH₂NH-L1-R, wherein n is a positive integer.

9. The method of claim 1, wherein R is a colloid polymer.

10. The method of claim 1, wherein R comprises a polyalkylene oxide.

11. The method of claim 10, wherein the polyalkylene oxide is polyethylene glycol (PEG).

12. The method of claim 10, wherein the polyethylene glycol has a weight average molecular weight of at least about 350.

13. The method of claim 11, wherein the polyethylene glycol has a weight average molecular weight of from about 750 to about 20,000.

14. The method of claim 13, wherein the polyethylene glycol has a weight average molecular weight of from about 1,000 to about 5,000.

15. The method of claim 1, wherein the cell is a malignant cell.

16. The method of claim 15, wherein the malignant cell is a cancer cell selected from the group consisting of prostate, breast, pancreatic, lung, renal, ovarian, skin, cervical, glioblastoma, brain, osteosarcoma, head and neck, colorectal, testicular, thyroid, leukemia, and myeloma.

17. The method of claim 1, wherein the cell is contacted with the conjugate of formula (I) in vitro.

18. The method of claim 1, wherein the cell is part of a mammal, and the conjugate is administered to the mammal as part of a pharmaceutically acceptable dosage form.

19. The method of claim 18, wherein the pharmaceutically acceptable dosage form is a parenteral dosage form.

20. The method of claim 19, wherein the amount of the conjugate administered to the mammal is from about 0.01 to about 1000 mg/kg.

21. The method of claim 20, further comprising administering an additional pharmaceutically active agent to the mammal.

22. The method of claim 21, wherein the pharmaceutically active agent is an anticancer agent.

23. (canceled)

24. A conjugate of the formula: 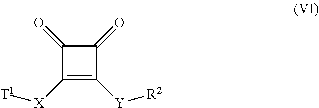
wherein T¹ is C₁₀-C₂₀ alkane, or C₁₀-C₂₀ unsaturated alkane (1-2 unsaturation, each independently Z or E)
X and Y are independently O, NR¹;
R¹ is H, C₁-C₆ alkyl, C₃-C₁₂ cycloalkyl, C₃-C₁₂ heterocycloalkyl with 1-3 heteroatoms, or OH;
R²= 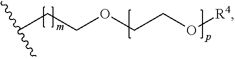
wherein m is an integer from 1 to 8; p is an integer from 20 to 25 and R⁴ is H, 1-7 atom alkyl, branched alkyl, cycloalkyl or heterocycloalkyl, or R² is branched PEG chain MW<1500 or other polymer.
